SEQUENCE LISTING (15 sequences)

SEQ ID NO: 1 - protein, 4 aa, synthetic construct
  REGION 1..4: note = flexible glycine-serine
GGGS

SEQ ID NO: 2 - protein, 12 aa, synthetic construct
  REGION 1..12: note = flexible glycine-serine
GGGSGGGSGGGS

SEQ ID NO: 3 - protein, 7 aa, synthetic construct
  REGION 1..7: note = rigid alpha-helical linker
AEAAAKA

SEQ ID NO: 4 - protein, 15 aa, synthetic construct
  REGION 1..15: note = linker
GGGGSGGGGSGGGGS

SEQ ID NO: 5 - protein, 30 aa, synthetic construct
  REGION 1..30: note = linker
GGGGSGGGGSGGGGSGGGGSGGGGSGGGGS

SEQ ID NO: 6 - protein, 45 aa, synthetic construct
  REGION 1..45: note = linker
GGGGSGGGGSGGGGSGGGGSGGGGSGGGGSGGGGSGGGGSGGGGS

SEQ ID NO: 7 - protein, 60 aa, synthetic construct
  REGION 1..60: note = linker
GGGGSGGGGSGGGGSGGGGSGGGGSGGGGSGGGGSGGGGSGGGGSGGGGSGGGGSGGGGS

SEQ ID NO: 8 - protein, 5 aa, synthetic construct
  REGION 1..5: note = linker
GGGGS

SEQ ID NO: 9 - protein, 10 aa, synthetic construct
  REGION 1..10: note = linker
GGGGSGGGGS

SEQ ID NO: 10 - protein, 20 aa, synthetic construct
  REGION 1..20: note = linker
GGGGSGGGGSGGGGSGGGGS

SEQ ID NO: 11 - protein, 25 aa, synthetic construct
  REGION 1..25: note = linker
GGGGSGGGGSGGGGSGGGGSGGGGS

SEQ ID NO: 12 - protein, 35 aa, synthetic construct
  REGION 1..35: note = linker
GGGGSGGGGSGGGGSGGGGSGGGGSGGGGSGGGGS

SEQ ID NO: 13 - protein, 40 aa, synthetic construct
  REGION 1..40: note = linker
GGGGSGGGGSGGGGSGGGGSGGGGSGGGGSGGGGSGGGGS

SEQ ID NO: 14 - protein, 50 aa, synthetic construct
  REGION 1..50: note = linker
GGGGSGGGGSGGGGSGGGGSGGGGSGGGGSGGGGSGGGGSGGGGSGGGGS

SEQ ID NO: 15 - protein, 55 aa, synthetic construct
  REGION 1..55: note = linker
GGGGSGGGGSGGGGSGGGGSGGGGSGGGGSGGGGSGGGGSGGGGSGGGGSGGGGS

(19) United States
(12) Patent Application Publication
(10) Pub. No.: US 2024/0060963 A1
(43) Pub. Date: Feb. 22, 2024

(54) COMPOSITIONS FOR DETECTING SECRETION AND METHODS OF USE

(71) Applicants: THE GENERAL HOSPITAL CORPORATION, Boston, MA (US); THE BROAD INSTITUTE, INC., Cambridge, MA (US)

(72) Inventors: Sean Burns, Boston, MA (US); Jason Wright, Cambridge, MA (US); Thomas Sundberg, Cambridge, MA (US)

(73) Assignees: THE GENERAL HOSPITAL CORPORATION, Boston, MA (US); THE BROAD INSTITUTE, INC., Cambridge, MA (US)

(21) Appl. No.: 18/446,945

(22) Filed: Aug. 9, 2023

Related U.S. Application Data
(62) Division of application No. 16/606,028, filed on Oct. 17, 2019, now abandoned.
(60) Provisional application No. 62/486,807, filed on Apr. 18, 2017.

(51) Int. Cl. G01N 33/50; A61P 35/00; A61K 35/17; C07K 14/705; C12N 5/0783; C12N 15/10; C12N 15/11; C12N 15/62; G01N 21/64; G01N 33/569; G01N 33/58

(52) U.S. Cl. CPC G01N 33/505; A61P 35/00; A61K 35/17; C07K 14/705; C12N 5/0636; C12N 15/1093; C12N 15/11; C12N 15/62; G01N 21/6428; G01N 33/502; G01N 33/56972; G01N 33/582; C12N 2310/20; C07K 2319/03; C12N 2510/00; G01N 2021/6439

(57) ABSTRACT

The present invention provides methods and compositions based on a non-naturally occurring nucleic acid construct encoding a fusion protein for quantitating levels of secretion in a single cell which may comprise a protein sequence which may comprise a cytoplasmic domain, a transmembrane domain and a vesicular domain, wherein the vesicular domain may comprise a protein tag sequence, wherein upon expression of the fusion protein by a cell, the fusion protein localizes to the membrane of a secretory vesicle such that the protein tag localizes to the lumen of the secretory vesicle, and wherein the protein tag binds to a cell-impermeable marker; whereby upon secretion of the contents of the secretory vesicle, the protein tag is exposed to the cell-impermeable marker, the fusion protein is recycled back into the cell, and the single cell becomes labeled with the marker relative to the amount of secretion.

### RELATED APPLICATIONS AND INCORPORATION BY REFERENCE

This application claims priority to and benefit of U.S. Provisional Patent Application 62/486,807 filed Apr. 18, 2017.

The foregoing applications, and all documents cited therein or during their prosecution (“appln cited documents”) and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The instant application contains a Sequence Listing which has been filed electronically in eXtensible Markup Language (XML) format and is hereby incorporated by reference in its entirety. Said XML copy, created on Oct. 31, 2023, is named BN00007_0385_SL.xml and is 20 KB in size.

### FIELD OF THE INVENTION

The present invention provides methods and compositions for quantitating secretion in single cells and for sorting cells based on secretion.

### BACKGROUND OF THE INVENTION

Peptide hormones, cytokines and neuropeptides are signaling molecules that play key roles in normal physiology and disease states. Many diseases result from altered secretion of proteins, enzymes and signaling molecules, such as too little insulin in diabetes mellitus or too much cytokine release in autoimmune conditions. Moreover, genes regulating secretion are valuable potential drug targets (e.g., GLP1 receptor on pancreatic beta cells, NAV1.7 sodium channel on pain-sensing nerves).

Traditional immunoassays for these secreted proteins, such as the enzyme-linked immunosorbent assay (ELISA) and radioimmunoassay, have enabled limited investigation into the pathways regulating their secretion, yet these assays are too expensive and time consuming to be useful for large-scale genome-wide chemical and genetic screening. For example, immunoassays for secreted molecules of interest require suitable antibodies and enzyme substrate conversion assays offer only indirect functional measurements of secreted substances. Additionally, previous assays are not applicable to pooled screening options, limiting the applicability of modern genetic tools (e.g., CRISPR nuclease and CRISPR transactivator reagents). Thus, comprehensive interrogation of genes controlling secretion is impractical with existing assays.

A particular secreted peptide of interest is insulin. Failure to maintain adequate insulin secretion is central to the pathogenesis of both type 1 and type 2 diabetes. Determining the genetic pathways that regulate insulin secretion and finding small molecule probes of these pathways would greatly advance our understanding of the beta cell and bring us closer to a cure for both forms of diabetes. However, high throughput screens of insulin secretion using genetic (e.g., RNAi, CRISPR) or chemical perturbations are currently impracticable due to the lack of an amenable assay for measuring secreted insulin. Insulin ELISA kits and radioimmunoassays are not well suited to this application due to their expense, complicated handling requirements and restriction to 96-well format. Thus, a need exists for a high-throughput method of tracking peptide secretion, in particular, insulin hormone secretion.

Citation or identification of any document in this application is not an admission that such document is available as prior art to the present invention.

### SUMMARY OF THE INVENTION

It is an object of the present invention to provide compositions and methods for quantitating secretion from single cells. It is another object of the present invention to provide for sorting cells based on quantitated secretion levels. It is another object of the present invention to provide for cost effective and quantitative high throughput screens for determining pathways and genes required for secretion. It is another object of the present invention to provide for cost effective and quantitative high throughput screens for agents capable of modulating secretion. It is another object of the present invention to provide for pooled screens.

In a first aspect, the present invention provides for a non-naturally occurring nucleic acid construct encoding a fusion protein for quantitating levels of secretion in a single cell which may comprise a protein sequence which may comprise a cytoplasmic domain, a transmembrane domain and a vesicular domain, wherein the vesicular domain incorporates a protein tag sequence, wherein upon expression of the fusion protein by a cell, the fusion protein localizes to the membrane of a secretory vesicle such that the protein tag localizes to the lumen of the secretory vesicle, and wherein the protein tag binds to a cell-impermeable marker; whereby upon secretion of the contents of the secretory vesicle, the protein tag is exposed to the cell- impermeable marker, the fusion protein is recycled back into the cell, and the single cell becomes labeled with the marker relative to the amount of secretion. The cell impermeable marker may be present in the extracellular space of cells expressing the fusion protein. The cell impermeable marker may be added to tissue culture media or injected into an animal. The cell-impermeable marker may be a fluorescent marker. The tag that binds to the cell-impermeable marker may be a commercially available tag. The commercially available tag may be a SNAP-tag. The cell-impermeable marker may be a commercially available fluorescent marker. The fluorescent marker may be the SNAP Surface substrate. The protein sequence which may comprise a cytoplasmic domain, a transmembrane domain and a vesicular domain may be, or be derived from, a vesicle membrane protein. The domains may be derived from a combination of domains from vesicle membrane proteins. Not being bound by a theory, the general concept of the present invention may be accomplished with any combination of cytoplasmic domain, transmembrane domain, and vesicular domain that when expressed ectopically in a cell localizes to a secretory vesicle. Not being bound by a theory, expression of a fusion protein in a cell where only a small fraction of the fusion protein localizes to a secretory vesicle would still allow detection of secretion according to the present invention. The vesicle membrane protein may comprise VAMP1, VAMP2, VAMP3, VAMP4, VAMPS, VAMP7, VAMPS, synaptophysin or a synaptotagmin family protein.

The nucleic acid construct may further comprise a regulatory sequence operably linked to the nucleic acid construct encoding a fusion protein. The regulatory sequence may allow for inducible expression of the fusion protein. The regulatory sequence may allow for tissue specific expression of the fusion protein. Tissue specific expression is advantageous when the fusion protein is expressed in a multicellular organism, such as an animal model.

The nucleic acid construct may further comprise a selective marker operably linked to a second regulatory sequence. The selective marker may be used to select for cells that express the fusion protein from the nucleic acid construct. The selective marker may be an antibiotic resistance gene.

In another aspect, the present invention provides for a fusion protein encoded by any nucleic acid construct described herein. The fusion protein may be modified in order to have increased expression or increased efficiency of detecting secretion. The fusion protein may comprise a cytoplasmic domain that has at least 90% identity to the amino acid sequence of a cytoplasmic domain of VAMP1, VAMP2, VAMP3, VAMP4, VAMPS, VAMP?, VAMPS, synaptophysin or a synaptotagmin family protein. The fusion protein may comprise a transmembrane domain that has at least 90% identity to the amino acid sequence of a transmembrane domain of VAMP1, VAMP2, VAMP3, VAMP4, VAMPS, VAMP?, VAMPS, synaptophysin or a synaptotagmin family protein. The fusion protein may comprise a vesicular domain that has at least 90% identity to the amino acid sequence of a vesicular domain of VAMP1, VAMP2, VAMP3, VAMP4, VAMPS, VAMP?, VAMPS, synaptophysin or a synaptotagmin family protein. The fusion protein may comprise a protein sequence which may comprise a cytoplasmic domain, a transmembrane domain and a vesicular domain that has at least 90% identity to the amino acid sequence of VAMP1, VAMP2, VAMP3, VAMP4, VAMPS, VAMP?, VAMPS, synaptophysin or a synaptotagmin family protein.

In another aspect, the present invention provides for a cell which may comprise any nucleic acid construct described herein, wherein the cell is capable of expressing the encoded fusion protein. The cell may be an endocrine cell, an exocrine cell, an immune cell, a hematopoietic cell, a neuron, a hepatocyte, a myocyte, a kidney cell, an adipocyte, an osteocyte, a stem cell or a cell line derived therefrom. The endocrine cell may be a beta cell, an alpha cell, an L cell, a K cell, other endocrine cell or a cell line derived therefrom. The immune cell may be a B cell, a T cell, a CAR T cell, a natural killer cell, a monocyte, a macrophage, a plasma cell, a dendritic cell, a mast cell, a neutrophil or a cell line derived therefrom. The cell may be an embryonic stem cell, an adult stem cell, or an iPS cell. The cell may further comprise a nucleic acid encoding a CRISPR enzyme.

In another aspect, the present invention provides for a eukaryotic organism which may comprise a cell as described herein. The eukaryotic organism is preferably a transgenic animal. The transgenic animal may be an animal model. The animal model may be an animal model of disease. The disease may be a disease where there is abnormal secretion. In one embodiment, the animal model is a model of diabetes. Not being bound by a theory, secretion in response to a treatment may be efficiently determined by expression of the fusion protein of the present invention in an animal model.

In another aspect, the present invention provides for a method of screening for modulators of secretion which may comprise: contacting any cell described herein with a test compound in the presence of a cell-impermeable marker capable of binding to the fusion protein tag; and determining fluorescence of the cell, whereby a difference in fluorescence as compared to the cell not contacted with a test compound indicates that the test compound is a modulator of secretion. The fluorescence may be increased or decreased as compared to the cell not contacted. The method may further comprise treating the cell with a secretagogue. Not being bound by a theory, in order to screen for a modulator of secretion, the cells need to be stimulated for secretion prior to or simultaneously with addition of a test compound. The determining of fluorescence of single cells may be by cell sorting.

In another aspect, the present invention provides for a method of pooled screening for modulators of secretion which may comprise: introducing a library which may comprise two or more test compounds to a population of cells which may comprise any cell described herein in the presence of a cell-impermeable marker capable of binding to the fusion protein tag, wherein the test compounds in the library can be identified by sequencing; sorting the population of cells into groups which may comprise at least one cell of the population, wherein the sorting is based on differences in fluorescence in each cell in the population of cells, and wherein fluorescence correlates to the amount of secretion; determining the test compounds introduced for each sorted group by sequencing, whereby a difference in fluorescence as compared to a cell contacted with a control test compound or not contacted with a test compound indicates that the test compound is a modulator of secretion. The method may further comprise treating the cell with a secretagogue to stimulate secretion.

The test compound in any method of the present invention may be a test nucleic acid. The test nucleic acid may comprise a unique barcode sequence. Not being bound by a theory, the identity of a test nucleic acid introduced to individual cells may be determined by sequencing the barcode. In preferred embodiments, a single test nucleic acid is introduced to a single cell, such that each cell receives only a single test nucleic acid. The test nucleic acid may comprise a CRISPR guide RNA, RNAi or gene expression sequence. The test nucleic acid may comprise a nucleotide sequence encoding for a CRISPR enzyme and a nucleotide sequence encoding for a CRISPR guide RNA. In one embodiment, test nucleic acids that allow expression or knockdown of genes are introduced into a population of cells expressing the fusion protein. Not being bound by a theory, each cell is an individual experiment. The present invention provides the advantage of being able to perform an experiment in a pooled population of cells due to the fact that the fluorescent signal is preserved in each individual cell and is not secreted into the culture media. Because the fusion protein of the present invention results in a quantifiable increase in fluorescence in each individual cell, the cells may be sorted based on the signal and each cell may be analyzed for the barcode associated with the test nucleic acid introduced. In one embodiment, the test nucleic acid is a plasmid. In another embodiment, the test nucleic acid is a vector. The vector may be a viral vector. The viral vector may be an adenovirus, lentivirus, adeno associated virus (AAV), herpesvirus, or pox virus. Methods of introducing the test nucleic acid may be, but not limited to transfection or transduction.

In another aspect, the present invention provides for a method of sorting T cells which may comprise: contacting a population which may comprise two or more T cells with a sample which may comprise at least one antigen; and sorting the population of cells into groups which may comprise at least one cell of the population, wherein the sorting is based on differences in fluorescence in each cell in the population of cells, and wherein fluorescence correlates to the amount of secretion of cytokines; whereby a group with increased fluorescence as compared to the population of cells indicates that the T cells within that group is reactive to the antigen. The T cells may be CAR T cells and the cells may be sorted based on binding of chimeric antigen receptors to an antigen.

In another aspect, the present invention provides for a method of preparing a pharmaceutical composition for treating a patient in need thereof which may comprise: introducing any of the nucleic acid constructs or fusion proteins described herein to a population which may comprise two or more T cells obtained from the patient; contacting the population of T cells with a sample which may comprise at least one antigen in the presence of a cell-impermeable marker capable of binding to the fusion protein tag; sorting the population of cells into groups which may comprise at least one cell of the population, wherein the sorting is based on differences in fluorescence in each cell in the population of cells, and wherein fluorescence correlates to the amount of secretion of cytokines; and preparing cells by a method which may comprise: (i) determining T cell receptor pairs expressed by T cells for at least one sorted group and generating at least one CAR T cell expressing a T cell receptor pair determined from the group; or (ii) expanding T cells for at least one sorted group, wherein the group has high fluorescence. The patient in need thereof may be suffering from cancer. The sample which may comprise at least one antigen may be a tumor sample.

In another aspect, the present invention provides for a pharmaceutical composition prepared by any method described herein.

In another aspect, the present invention provides for a method of treatment which may comprise administering any pharmaceutical composition described herein to the patient in need thereof.

In another aspect, the present invention provides for a kit which may comprise any nucleic acid construct described herein, a cell-impermeable marker capable of binding to the tag sequence, and instructions for use.

In another aspect, the present invention provides for a kit which may comprise any cell described herein, and instructions for use.

A kit of the present invention may further comprise at least one nucleic acid construct encoding a CRISPR guide RNA.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention. Nothing herein is intended as a promise.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1 illustrates a fundamental aspect of the present invention. Cells expressing the SNAP-tagged synaptobrevin protein become increasingly fluorescent as they secrete more of the substance of interest through the regulated pathway.

FIG. 2 provide data illustrating that cellular fluorescence increases with glucose stimulation; (top) 2.8 mM glucose, (bottom) 16.7 mM glucose.

FIG. 3 provides graphs showing that the intensity of cellular fluorescence is proportional to the degree of glucose stimulation (left), and correlates well with the amount of insulin (INS) secreted (right).

FIG. 4 provides data from an isolated clonal cell line with improved response, including a 7-fold increase in fluorescence in high (16.7 mM, right) vs. low (2.8 mM, left) glucose conditions (identical gates).

FIG. 5 illustrates the schematic structure of the synaptoSNAP construct and the acquisition of fluorescence in the presence of the substrate.

FIG. 6 shows graphical results of a screen where INS1E cells expressing the reporter were treated with glucose in the presence and absence of sgRNAs. Depicted is the fluorescence distribution (listed as “PE-Texas Red-A”) of INS1E rat pancreatic beta cells in high glucose without any sgRNA/CRISPR-Cas9 treatment (left) and in the presence of 6468 sgRNAs/CRISPR-Cas9 targeting 1078 genes (right).

FIG. 7 provides a graph showing the results of sequencing of genomic DNA from the loss of INS secretion fraction from the screen in FIG. 6. Counts of each sgRNA in the loss of INS secretion fraction is plotted against their relative abundance in the initial library. The black line represents the expected counts relative to library. SgRNAs above the line showed a higher representation in the loss of INS secretion fraction than expected.

FIG. 8 provides a graph showing the results of sequencing of genomic DNA from the increase of INS secretion fraction from the screen in FIG. 6. Counts of each sgRNA in the increase of INS secretion fraction is plotted against their relative abundance in the initial library. The black line represents the expected counts relative to library. SgRNAs above the line showed a higher representation in the increase of INS secretion fraction than expected

FIGS. 9A-9C provides graphs depicting measurements of multiple sgRNAs for each gene from the screen in FIG. 6 collapsed into a single gene score by RIGER, which ranks sgRNAs according to their differential effects between two classes of samples, then identifies the genes targeted by the sgRNAs at the top of the list. (Fig. A,B) Genes that displayed a consistent 2-fold or greater enrichment score across sgRNA RIGER scores for each fraction were identified as candidate INS secretion genes. (Fig. C) The majority of genes identified in each fraction are specific to either increase or loss of INS secretion, with only 7/1078 (0.6%) of genes enriched in both fractions.

### DETAILED DESCRIPTION OF THE INVENTION

The present invention is based in part upon methods and compositions relating to detecting secretion by a cell, including but not limited to secretion of proteins, enzymes, neurotransmitters and chemicals, for example, digestive enzymes, cytokines, hormones, and surfactants.

The compositions and methods are useful in screening for compounds, molecules, genes, or genetic elements that modulate secretion. In preferred embodiments, the present invention provides for the high throughput measurement of secretion in the setting of genetic and chemical perturbations, and as such is well suited to screens for genes and compounds impacting physiologic processes.

The compositions and methods of the present invention provide for the rapid, and massively high throughput measurement of secretion from pools of cells. The present invention advantageously provides for two aspects previously not offered. (1) It can be applied to any secretory cell system both in vitro and in vivo and (2) it does not require separating and analyzing each cell individually but instead can connect secretory measurements to individually identifiable cells from the pool. Thus, the present invention has many applications in basic research and clinical/therapeutic discovery.

The compositions and methods of the present invention also provide for sorting T cells and for producing therapeutic compositions. The present invention advantageously provides for sorting T cells based on reactivity to an antigen, preferably to a tumor.

One embodiment of the present invention as provided herein is a system to accurately track the flux of vesicles using a tagged version of a protein present in the synaptic vesicle membrane (“synaptoSNAP”). One of ordinary skill in the art can appreciate that the present invention can utilize any protein known in the art that can localize to a synaptic vesicle membrane and has a domain present within the lumen of the vesicle. The present invention may utilize any protein previously undiscovered at the time of the present invention with these characteristics. The present invention may also utilize recombinant DNA technology to modify proteins and generate hybrid proteins to achieve the required characteristics.

In some embodiments, the protein is VAMP1, VAMP2, VAMP3, VAMP4, VAMPS, VAMP?, VAMPS, a Synaptotagmin, or Synaptophysin. Preferred sequences may be found at the website of the National Center for Biotechnology (NCBI) or at www.uniprot.org. UniProt describes each of the proteins and isoforms of the proteins or alternative sequences, as well as listing similar proteins in other organisms. All such sequences are intended to be included for use in the present invention. The protein may be derived from any organism with a homologous protein. In preferred embodiments, the protein is derived from an animal, including, but not limited to primates, such as humans, monkeys, or chimpanzee; rodents, such as mouse or rat; amphibians, such as frogs; zebrafish, insects, cats, dogs, cattle, horses or chickens. Non-mammalian versions of the fusion protein of the present invention are possible. Secretion in plants using a similar construct is within the scope of the present invention. Homologues may be determined using any sequence known in the art encoding for a vesicle membrane protein and performing a BLAST search, for example at the website of the National Center for Biotechnology (NCBI). The protein used to localize to a membrane vesicle may be shorter or longer than the endogenous protein. The protein may be modified for increased expression. The protein may be codon optimized. Not being bound by a theory, only the functional domains required for localizing to a secretory vesicle membrane is required for the present invention. The nucleotide sequence encoding the fusion protein of the present invention may have at least 50%, 60%, 70%, 75%, 80%, 85%, 90%, 95%, or 100% sequence identity to the nucleic acid sequence encoding the endogenous proteins. The proteins may have at least 50%, 60%, 70%, 75%, 80%, 8S%, 90%, 9S%, or 100% sequence identity to the amino acid sequence of the endogenous proteins. The protein may be a hybrid of more than one secretory vesicle membrane protein. The protein may be further mutated to delete non-essential regions. In one embodiment, the non-membrane spanning domain is deleted or truncated. In some proteins this may be a truncation of the N-or C-terminus. Not being bound by a theory, only domains required for inserting into a secretory vesicle membrane and for displaying the fusion protein tag when the secretory vesicle is exposed to the extracellular space is necessary.

As used herein, “vesicle membrane protein” refers to any vesicle associated membrane protein. That is any protein that localizes to the membrane of a vesicle. SNARE proteins or vesicle associated membrane proteins (VAMP) are exemplary vesicle membrane proteins that have a similar structure and are mostly involved in vesicle fusion. The VAMP proteins have an N-terminal cytoplasmic domain, a transmembrane domain, and a C-terminal vesicular domain. Such domains and sequences may be found online at www.uniprot.org. VAMP1 and VAMP2 proteins (synaptobrevins) are expressed in the brain and are constituents of the synaptic vesicles, where they participate in neuromediator release. VAMP3 (known as cellubrevin) is ubiquitously expressed and participates in regulated and constitutive exocytosis as a constituent of secretory granules and secretory vesicles. VAMPS and VAMP7 (SYBL1) participate in constitutive exocytosis. VAMPS is a constituent of secretory vesicles, myotubes and tubulovesicular structures. VAMP7 is found both in secretory granules and endosomes. VAMP8 (known as endobrevin) participates in endocytosis and is found in early endosomes. VAMP8 also participates the regulated exocytosis in pancreatic acinar cells. VAMP4 is involved in transport from the Golgi. The synaptotagmin gene family encodes proteins that mediate membrane trafficking in synaptic transmission and are described online at www.uniprot.org/uniprot/?query=family%3A%22synaptotagmin+family%22&sort=score. Exemplary family members have vesicular domains, transmembrane domains, and cytoplasmic domains. For example, Synaptotagmin-6 and -3, have an N-terminal vesicular domain, a transmembrane domain, and a C-terminal cytoplasmic domain. Extended synaptotagmin-1 (ESYT1) has C-terminal and N-terminal cytoplasmic domains, two transmembrane domains, and a lumenal domain. Synaptophysin (SYP) is described online at www.uniprot.org/uniprot/P08247#showFeatures. Synaptophysin contains three cytoplasmic domains, four transmembrane domains, and two vesicular domains. Any combination of these domains may be used in the present invention. Not being bound by a theory, a fusion protein which may comprise any vesicle membrane protein ectopically expressed in a cell will localize to secretory vesicles. Not being bound by a theory, a fusion protein which may comprise any combination of the domains of any vesicle membrane protein ectopically expressed in a cell will localize to secretory vesicles.

In preferred embodiments, the protein is synaptobrevin (a.k.a., vesicle-associated membrane protein-2, or VAMP2). The C-terminus of this trans-membrane protein resides within the lumen of the vesicles, and becomes exposed to the extracellular environment when the vesicle fuses to the cell membrane during exocytosis.

The vesicle membrane protein, preferably, synaptobrevin, is fused to a tag that binds a substrate or marker when exposed to the extracellular environment. As used herein, the term “tag” refers to any additional nucleotide sequence encoding a protein domain or any additional amino acid sequence that forms a protein domain added to a fusion protein that allows the fusion protein to be distinguished or separated from other proteins. In certain embodiments, the tag allows visualization of the fusion protein. In some embodiments, the tag binds to a marker that is cell impermeable. In some embodiments, the marker binds the tag irreversibly. In preferred embodiments the marker is a fluorescent marker. Alternative fluorescent substrates may include SNAP-cell TMR-star, SNAP-cell 647-SIR, SNAP-surface 488, SNAP-surface 549, SNAP-surface 649, SNAP-surface Alexa Flour 546, SNAP-surface Alexa Flour 647, SNAP-surface Alexa Flour 488, SNAP-Wista Green, CLIP-surface 488, CLIP-surface 547, CLIP-surface 647, or Halo substrates. In alternative embodiments, the tag may be a Halo-tag, Halo-biotin tag, CLIP-tag, CLIP-biotin, SNAP-tag, or SNAP-biotin. In some embodiments, the fusion protein may have more than one tag. In preferred embodiments, the tag is a commercially available “SNAP tag” (New England Biolabs). The SNAP-tag is a small protein based on mammalian 0⁶-alkylguanine-DNA-alkyltransferase (AGT) (Keppler, A., et al., Proc Natl Acad Sci U S A. 2004 Jul. 6; 101(27):9955-9. Epub 2004 Jun. 28). SNAP-tag substrates are derivatives of benzyl purines and benzyl pyrimidines. In the labeling reaction, the substituted benzyl group of the substrate is covalently attached to the SNAP-tag. The SNAP-tag fluoresces only when it complexes with its substrate. The present invention utilizes a substrate that is cell impermeable such that the Synapto-SNAP only fluoresces upon fusion to the cell membrane, an event that precisely corresponds to the rate of vesicle secretion. Further, as vesicles recycle and compensatory endocytosis ensues, substrate-bound synaptoSNAP re-enters the cell, and fluorescence accumulates, such that the signal-to-noise ratio is enhanced over time.

The system of the present invention is amenable to a host of applications. At rest, cells expressing synaptoSNAP are non-fluorescent, with the protein residing primarily within secretory vesicles. Upon stimulation of secretion either through chemical treatment, genetic perturbation, metabolic stimuli or environmental changes, the protein travels with the vesicles to the cell surface, gets exposed to the extracellular environment during exocytosis, and covalently binds to the cell-impermeable, fluorescent (“SNAP Surface”) substrate that has been added to the media. Through vesicle recycling and compensatory endocytosis, the now-fluorescent synaptoSNAP protein is incorporated into newly-formed, intracellular vesicles, contributing to an accumulation of fluorescence within the cells as secretion proceeds. Fluorescence intensity of the cells serves as a close proxy for secretion of the substance of interest in all cell models tested.

In one embodiment, the fusion protein is designed to utilize synaptobrevin in combination with the SNAP-tag and non-cell permeable substrates. However, alternative embodiments of the general concept include any macromolecule which closely fluxes to and from the cell membrane during exocytosis/endocytosis. Further embodiments include alternative detection systems that can be utilized to (i) multiplex different secretion mechanisms by different fluorophores, (2) use non-fluorescent substrates to isolate cells based on other properties (e.g., magnetic particles, mass, viability), and (3) identify substrates that are intracellular and measure their secretion from the cell through synaptoSNAP. Overall, the present invention can be applied to the current understanding of proteins that are involved in vesicle tracking to deliver a rapid detection system such as fluorescence to enable pooled screening on secretion phenotypes.

Fusion proteins may comprise a single continuous linear polymer of amino acids which may comprise the full or partial sequence of two or more distinct proteins. The construction of fusion proteins is well-known in the art. Two or more amino acids sequences may be joined chemically, for instance, through the intermediacy of a crosslinking agent. In a preferred embodiment, a fusion protein is generated by expression of a fusion gene construct in a cell. A fusion gene construct may comprise a single continuous linear polymer of nucleotides which encode the full or partial sequences of two or more distinct proteins. Fusion gene constructs generally also contain replication origins active in eukaryotic and/or prokaryotic cells and one or more selectable markers encoding, for example, drug resistance. They may also contain viral packaging signals as well as transcriptional and/or translational regulatory sequences and RNA processing signals. Fusion gene constructs of the present invention contain a gene that localizes to a secretory vesicle and a tag protein that is capable of binding a cell impermeable marker.

The present invention encompasses fusion proteins encoded by the nucleic acid constructs described herein. The fusion proteins have at least 70%, 75%, 80%, 85%, 90%, or 95% sequence identity to the fusion proteins encoded by the nucleic acid constructs of the present invention, such that the resulting fusion protein retains the ability to localize to a secretory vesicle and to bind a cell impermeable marker upon being brought into contact with such marker. In some embodiments, the fusion proteins have at least 90%, 91%, 92%, 93%, 94%, 95%, 96%, 97%, 98%, or 99% sequence identity to the fusion proteins encoded by the nucleic acid constructs of the present invention.

In one embodiment, nucleic acid construct can be designed to be inducible. The nucleic acid construct may be inducible through a provided signal such as, but not limited to tet-ON, thus, enabling precise time courses for secretion measurement. Further the system can be driven by cell type specific promoters (Chen et al., TiProD: the Tissue-specific Promoter Database, Nucleic Acids Research, 2006). This would enable tissue specific secretory phenotypes to be measured, particularly useful in animal model or co-culture experimental models.

A nucleic acid construct sequence encoding the fusion protein according to the invention as described herein can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the U6 promoter. The fusion gene constructs may also contain other transcriptional and/or translational regulatory sequences to drive expression of the fusion gene.

The invention provides that at least one switch may be selected from the group consisting of antibiotic based inducible systems, electromagnetic energy based inducible systems, small molecule based inducible systems, nuclear receptor based inducible systems and hormone based inducible systems. In a more preferred embodiment the at least one switch may be selected from the group consisting of tetracycline (Tet)/DOX inducible systems, light inducible systems, ABA inducible systems, cumate repressor/operator systems, 4OHT/estrogen inducible systems, ecdysone-based inducible systems and FKBP12/FRAP (FKBP12-rapamycin complex) inducible systems.

In one embodiment the nucleic acid construct encoding the fusion protein of the present invention is stably integrated into a cell and provides for long-term measurements of secretion in cells and in vivo. In other embodiments, the system can be introduced transiently for high-turnover, short term experiments. This is particularly useful for primary cell types and non-dividing cell types in which long term experimentation is not feasible.

Any of the assays described herein may utilize more than one fusion protein of the present invention. Two or more fusion proteins may be fused to different tags for binding to different markers. Two or more fusion proteins may utilize different secretory vesicle membrane bound proteins. Such embodiments are particularly useful in animal model or co-culture experimental models.

The present invention is also applicable to the determination of secretion in a multicellular organism, such as an animal model. The fusion protein may be expressed from a transgene within a cell of the multicellular organism. The multicellular organism may be a transgenic animal that expresses the fusion protein of the present invention from a transgene integrated into the host genome. In one embodiment, secretion is tracked in an animal. The animal may be a mammal. In preferred embodiments, a rodent capable of expressing the fusion protein of the present invention is used. The animal may be a mouse model. The mouse model may be a model of a disease that has a defect in secretion. In one embodiment the animal model is an animal model of diabetes. The fusion protein may be under the control of a cell or tissue specific promoter. Not being bound by a theory, a mouse model may express the fusion protein in beta cells and secretion may be monitored by administering the cell impermeable marker to the mouse and quantifying the amount of marker internalized by beta cells in the mouse upon contacting the mouse with a test compound. The internalization of the marker may be visualized using modern imaging technology.

It will be appreciated that where reference is made to a method of modifying an organism or mammal including human or a non-human mammal or organism by insertion of a transgene, this may apply to the organism (or mammal) as a whole or just a single cell or population of cells from that organism (if the organism is multicellular). Applicants envisage, inter alia, a single cell or a population of cells and these may preferably be modified ex vivo and then re-introduced, e.g., transplanted to make transgenic organisms that express the fusion protein in certain cells.

In one aspect, the present invention provides a transgenic eukaryote, e.g., mouse. In certain preferred embodiments, the transgenic eukaryote, e.g., mouse may comprise a transgene encoding the fusion protein of the present invention knocked into the Rosa26 locus. In one aspect, the present invention provides a transgenic eukaryote, e.g., mouse wherein the transgene is driven by the ubiquitous CAG promoter thereby providing for constitutive expression of the fusion protein in all tissues/cells/cell types of the mouse. In one aspect, the present invention provides a transgenic eukaryote, e.g., mouse wherein the transgene driven by the ubiquitous CAG promoter further may comprise a Lox-Stop-polyA-Lox (LSL) cassette thereby rendering fusion protein expression inducible by the Cre recombinase.

The eukaryotic cell may comprise a fusion protein transgene that is functionally linked to a constitutive promoter, or a tissue specific promoter, or an inducible promoter; and, the eukaryotic cell can be part of a non-human transgenic eukaryote, e.g., a non-human mammal, primate, rodent, mouse, rat, rabbit, canine, dog, cow, bovine, sheep, ovine, goat, pig, fowl, poultry, chicken, fish, insect or arthropod; advantageously a mouse. The isolated eukaryotic cell or the non-human transgenic eukaryote can express an additional protein or enzyme, such as Cre; and, the expression of Cre can be driven by coding therefor functionally or operatively linked to a constitutive promoter, or a tissue specific promoter, or an inducible promoter.

Transgenic non-human eukaryotic organisms, e.g., animals are also provided in an aspect of practice of the instant invention. Preferred examples include animals which may comprise the fusion protein, in terms of polynucleotides encoding the protein itself. In certain aspects, the invention involves a constitutive or conditional or inducible fusion protein non- human eukaryotic organism, such as an animal, e.g., a primate, rodent, e.g., mouse, rat and rabbit, are preferred; and can include a canine or dog, livestock (cow/ bovine, sheep /ovine, goat or pig), fish, fowl or poultry, e.g., chicken, and an insect or arthropod, with it mentioned that it is advantageous if the animal is a model as to a human or animal genetic disease or condition, such as a disease or disorder with abnormal secretion, such as diabetes, as use of the non-human eukaryotic organisms in genetic disease or condition modeling is preferred. To generate transgenic mice with the constructs, as exemplified herein one may inject pure, linear DNA into the pronucleus of a zygote from a pseudo pregnant female, e.g. a CB56 female. Founders may then be identified, genotyped, and backcrossed to CB57 mice. The constructs may then be cloned and optionally verified, for instance by Sanger sequencing. Knock-ins are envisaged (alone or in combination).

The fusion gene constructs may be introduced into cells by any method of nucleic acid transfer known in the art, including, but not limited to, viral vectors, transformation, co-precipitation, electroporation, neutral or cationic liposome-mediated transfer, microinjection or gene gun. Viral vectors include retroviruses, poxviruses, herpes viruses, adenoviruses, and adena-associated viruses (AAV). Particularly preferred in the present invention are retroviral vectors, which are capable of stable integration into the genome of the host cell. For example, retroviral constructs encoding integration and packaging signals, drug resistance markers and the fusion proteins described herein are useful in the practice of the invention.

The recombinant cells of the invention that express the fusion constructs of the invention provide for development of screening assays, particularly for high throughput screening of molecules that up-or down-regulate the activity of secretion.

Due to the redundancy of the genetic code, any fusion protein of the present invention could be specified by any number of nucleic acid sequences in which synonymous base changes have been incorporated. Therefore, the nucleic acid sequences described herein should be taken as case examples of one such instance for each fusion protein, rather than the only tolerated nucleic acid sequence. The amino acid sequence of each construct ultimately determines the function of the fusion protein, though many possible nucleic acid sequences can specify each the sequence of each peptide.

Traditional methods of investigating peptide secretion are time-intensive and expensive. ELISA tests are accurate only within a limited range of detection, and as such, results often need to be verified by using serial dilution assays to verify that the signal detected is within the linear range.

The present invention provides methods that are more efficient, cost effective, sensitive, accurate, and therefore more amenable to large-scale high throughput chemical and genetic screens than the standard methods known in the art to date.

Any screening modality known in the art can be used to screen for modulators of secretion in conjunction with the fusion protein of the present invention. For example, natural products libraries can be screened using assays of the invention. The present invention contemplates screens for synthetic small molecule agents, chemical compounds, chemical complexes, and salts thereof. Other molecules that can be identified using the screens of the invention include proteins and peptide fragments, peptides, nucleic acids and oligonucleotides, carbohydrates, phospholipids and other lipid derivatives. Other modulators of peptide secretion can also include genes or genetic elements that are involved in regulating the pathways that control hormone secretion.

In another aspect, synthetic libraries (Needels et al., Proc. Natl. Acad. Sci. USA 90:1 0700-4, 1993; Ohlmeyer et al., Proc. Natl. Acad. Sci. USA 90: 10922-10926, 1993; Lam et al., International Patent Publication No. WO 92/00252; Kocis et al., International Patent Publication No. WO 9428028) and the like can be used to screen for compounds that modulate vesicle secretion.

Test compounds are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., Tib Tech, 14:60, 1996).

RNAi and open reading frame (ORF) libraries, such as those of the RNAi Consortium at the Broad Institute, can be used to screen for genes that increase or decrease peptide secretion. Additionally, CRISPR libraries as described herein can be used to screen for genes that increase or decrease secretion.

The present invention also provides a nucleic acid expression vector which may comprise a nucleic acid sequence encoding a fusion protein described herein, such that when the vector is expressed by a cell, the fusion protein is localized to a secretory vesicle; and upon secretion by the cell the tag is exposed to a marker that becomes internalized to the cell after recycling of the fusion protein. Optionally, the nucleic acid expression vector may comprise a promoter, wherein the promoter is operatively linked to the nucleic acid sequence encoding the fusion protein. Optionally, the nucleic acid expression vector may comprise a selective marker operatively linked to a second promoter. The selective marker can be an antibiotic resistance gene, drug resistance gene, toxin resistance gene or a cell surface marker. Not being bound by a theory, cells stably expressing the fusion protein may be selected for by use of a selective marker.

Alternatively, a nucleic acid expression vector may comprise any nucleic acid construct described herein operatively linked to a promoter and a selective marker operatively linked to a second promoter.

As used herein, the terms “selectable marker” and “positive selection marker” refer to a gene encoding a product that enables only the cells that carry the gene to survive and/or grow under certain conditions. For example, plant and animal cells that express the introduced neomycin resistance (Neo (r)) gene are resistant to the compound G418. Cells that do not carry the Neo (r) gene marker are killed by G418. Other positive selection markers are known to or are within the purview of those of ordinary skill in the art.

The expression vector for introducing the fusion protein of the present invention into a host cell may additionally comprise one or more further polynucleotide(s) encoding one or more additional selectable marker(s). Accordingly, in one embodiment of the present invention selection with one or more different selection system(s) (e.g. antibiotic resistant selection systems such as neo/G418) can be applied to further improve the performance. Selectable markers include but are not limited to Blasticidin, Zeocin™, Puromycin, G418, Hygromycin, and Phleomycin. Besides further eukaryotic selectable markers, allowing the selection of eukaryotic host cells expressing the fusion protein of the present invention, also prokaryotic selectable markers can be used, which allow the selection in prokaryotic host cells. Examples of respective prokaryotic selectable markers are markers which provide a resistance to antibiotics such as e.g. ampicillin, kanamycin, tetracycline and/or chloramphenicol. Use of selectable markers allows the generation of stable cell lines that stably express the fusion protein of the present invention. In one embodiment, a stable cell line is generated without the use of selectable marker as described in U.S. Pat. No. 6,692,965, and International PCT Patent Application Publication No. WO 2005/079462 A2, incorporated herein by reference.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

The practice of the present invention employs, unless otherwise indicated, conventional techniques for generation of genetically modified mice. See Marten H. Hofker and Jan van Deursen, TRANSGENIC MOUSE METHODS AND PROTOCOLS, 2nd edition (2011).

“Compound” as used herein encompasses all types of organic or inorganic molecules, including but not limited to proteins, peptides, polysaccharides, lipids, nucleic acids, small organic molecules, inorganic compounds, and derivatives thereof.

The term “low” as used herein generally means lower by a statically significant amount; for the avoidance of doubt, “low” means a statistically significant value at least 10% lower than a reference level, for example a value at least 20% lower than a reference level, at least 30% lower than a reference level, at least 40% lower than a reference level, at least 50% lower than a reference level, at least 60% lower than a reference level, at least 70% lower than a reference level, at least 80% lower than a reference level, at least 90% lower than a reference level, up to and including 100% lower than a reference level (i.e. absent level as compared to a reference sample).

The term “high” as used herein generally means a higher by a statically significant amount relative to a reference; for the avoidance of doubt, “high” means a statistically significant value at least 10% higher than a reference level, for example at least 20% higher, at least 30% higher, at least 40% higher, at least 50% higher, at least 60% higher, at least 70% higher, at least 80% higher, at least 90% higher, at least 100% higher, at least 2-fold higher, at least 3-fold higher, at least 4-fold higher, at least 5-fold higher, at least 10-fold higher or more, as compared to a reference level.

The term “statistically significant” or “significantly” refers to statistical significance and generally means a two standard deviation below normal, or lower, concentration of the marker. The term refers to statistical evidence that there is a difference. It is defined as the probability of making a decision to reject the null hypothesis when the null hypothesis is actually true. The decision is often made using the p-value.

“Polypeptide,” “Protein,” and “Peptide” are used interchangeably to refer to amino acid chains in which the amino acid residues are linked by covalent peptide bonds. The amino acid chains can be of any length of at least two amino acids, including full-length proteins. Unless otherwise specified, the terms “polypeptide,” “protein, ” and “peptide” also encompass various modified forms thereof, including but not limited to glycosylated forms, phosphorylated forms, etc.

A secretagogue is a substance that causes another substance to be secreted from a cell.

“Fusion construct” refers to a non-naturally occurring hybrid or chimeric construct having two or more distinct portions covalently linked together, each portion being or being derived from a specific molecule. When two or more portions in a fusion construct as defined above are polypeptides and are linked together by peptide bonds, the fusion construct is conveniently referred to as “fusion protein.”

“Peptide hormones” are a class of peptides that are secreted into the blood stream and have endocrine functions in living animals.

“Fluorescent” molecules or moieties include those that are luminescent via a single electronically excited state, which is of very short duration after removal of the source of radiation. The wavelength of the emitted fluorescence light is longer than that of the exciting illumination (Stokes' Law), because part of the exciting light is converted into heat by the fluorescent molecule.

“Light” includes electromagnetic radiation having a wavelength of between about 300 nm and about 1100 nm, but can be of longer or shorter wavelength.

“Small molecule” includes compositions that have a molecular weight of less than about 5 kD and most preferably less than about 4 kD. Small molecules is, e.g., nucleic acids, peptides, polypeptides, peptidomimetics, carbohydrates, lipids or other organic or inorganic molecules.

“Heterologous gene” includes a gene that has been transfected into a host organism. Typically, a heterologous gene refers to a gene that is not originally derived from the transfected or transformed cells' genomic DNA.

“Recombinant nucleic acid molecules” include nucleic acid sequences not naturally present in the cell, tissue or organism into which they are introduced.

The term “operably linked” relates to the orientation of polynucleotide elements in a functional relationship. Operably linked means that the DNA sequences being linked are generally contiguous and, where necessary to join two protein coding regions, contiguous and in the same reading frame. However, since enhancers generally function when separated from the promoter by several kilobases, some nucleic acids are operably linked but not contiguous.

The terms “polynucleotide” and “nucleic acid molecule” are used interchangeably to refer to polymeric forms of nucleotides of any length. The polynucleotides may contain deoxyribonucleotides, ribonucleotides and/or their analogs. Nucleotides may have any three-dimensional structure, and may perform any function, known or unknown. The term “polynucleotide” includes single-, double-stranded and triple helical molecules.

“Oligonucleotide” refers to polynucleotides of between 5 and about 100 nucleotides of single-or double-stranded DNA. Oligonucleotides are also known as oligomers or oligos and are isolated from genes, or chemically synthesized by methods known in the art.

The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A nucleic acid molecule may also comprise modified nucleic acid molecules, such as methylated nucleic acid molecules and nucleic acid molecule analogs. Analogs of purines and pyrimidines are known in the art, and include, but are not limited to, aziridinycytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudouracil, 5-pentylnyluracil and 2,6-diaminopurine. The use of uracil as a substitute for thymine in a deoxyribonucleic acid is also considered an analogous form of pyrimidine.

The term “homologous” as used herein denotes a characteristic of a DNA sequence having at least about 70 percent sequence identity as compared to a reference sequence, typically at least about 85 percent sequence identity, preferably at least about 95 percent sequence identity, and more preferably about 98 percent sequence identity, and most preferably about 100 percent sequence identity as compared to a reference sequence. Homology is determined using, for example, a “BLASTN” algorithm. It is understood that homologous sequences can accommodate insertions, deletions and substitutions in the nucleotide sequence. Thus, linear sequences of nucleotides are essentially identical even if some of the nucleotide residues do not precisely correspond or align. The reference sequence is a subset of a larger sequence, such as a portion of a gene or flanking sequence, or a repetitive portion of a chromosome.

The term “transgenic cell” refers to a cell containing within its genome a nucleic acid encoding a fusion protein as described herein introduced by the any method of gene targeting.

The term “proliferating cell” includes any cell undergoing cell division.

A “host cell” includes an individual cell or cell culture that is or has been a recipient for vector(s) or for incorporation of nucleic acid molecules and/or proteins. Host cells include progeny of a single host cell, and the progeny may not necessarily be completely identical (in morphology or in total DNA complement) to the original parent due to natural, accidental, or deliberate mutation. A host cell includes cells transfected or transduced with the constructs of the present invention.

The term “modulates” as used herein refers to the decrease, inhibition, reduction, increase, or enhancement of a cellular process, gene function, expression, or activity.

Cells for use in the present invention may be any isolated cell or population of cells that are capable of secretion. Exemplary cell lines are described at the website of ATCC (www.atcc.org). Preferably, the cell can be treated with a secretogogue to induce the cell to secrete a biomolecule. Cells may be obtained from a subject, including but not limited to those of or derived from a particular organism, such as a mammal, including but not limited to human, or non-human eukaryote or animal or mammal as herein discussed, e.g., mouse, rat, rabbit, dog, livestock, or non-human mammal or primate. A eukaryotic organism may comprise cells as described herein. Cells may also be any immortalized tissue culture cell line available to those skilled in the art. Specific cell lines may be used depending on the secretory process being examined. For example, the cell may be an endocrine cell, an exocrine cell, an immune cell, a hematopoietic cell, a neuron, a hepatocyte, a myocyte, a kidney cell, an adipocyte, an osteocyte, a stem cell or a cell line derived therefrom. The endocrine cell may be a beta cell, an alpha cell, an L cell, a K cell, other endocrine cell or a cell line derived therefrom. The immune cell may be a B cell, a T cell, a CAR T cell, a natural killer cell, a monocyte, a macrophage, a plasma cell, a dendritic cell, a mast cell, a neutrophil or a cell line derived therefrom. The cell may be an embryonic stem cell, an adult stem cell, or an iPS cell.

The present invention may also include barcoding. Barcoding may be performed based on any of the compositions or methods disclosed in patent publication WO 2014047561 A1, Compositions and methods for labeling of agents, incorporated herein in its entirety. In one embodiment each test nucleic acid is associated with a barcode. The barcode may be a part of a vector used to introduce the test nucleic acid. In one embodiment, a sgRNA has a barcode. Sequencing of the barcode can be used to determine enrichment or depletion of test nucleic acids after sorting cells. Additionally, test nucleic acids from single cells can be determined if single cells are sequenced.

The term “barcode” as used herein, refers to any unique, non-naturally occurring, nucleic acid sequence that may be used to identify the originating source of a nucleic acid fragment. Such barcodes may be sequences including but not limited to, TTGAGCCT, AGTTGCTT, CCAGTTAG, ACCAACTG, GTATAACA or CAGGAGCC. Although it is not necessary to understand the mechanism of an invention, it is believed that the barcode sequence provides a high-quality individual read of a barcode associated with a viral vector, labeling ligand, shRNA, sgRNA or cDNA such that multiple species can be sequenced together.

DNA barcoding is also a taxonomic method that uses a short genetic marker in an organism's DNA to identify it as belonging to a particular species. It differs from molecular phylogeny in that the main goal is not to determine classification but to identify an unknown sample in terms of a known classification. Kress et al., “Use of DNA barcodes to identify flowering plants” Proc. Natl. Acad. Sci. U.S.A. 102(23):8369-8374 (2005). Barcodes are sometimes used in an effort to identify unknown species or assess whether species should be combined or separated. Koch H., “Combining morphology and DNA barcoding resolves the taxonomy of Western Malagasy Liotrigona Moure, 1961” African Invertebrates 51(2): 413-421 (2010); and Seberg et al., “How many loci does it take to DNA barcode a crocus?” PLoS One 4(2):e4598 (2009). Barcoding has been used, for example, for identifying plant leaves even when flowers or fruit are not available, identifying the diet of an animal based on stomach contents or feces, and/or identifying products in commerce (for example, herbal supplements or wood). Soininen et al., “Analysing diet of small herbivores: the efficiency of DNA barcoding coupled with high-throughput pyrosequencing for deciphering the composition of complex plant mixtures” Frontiers in Zoology 6:16 (2009).

It has been suggested that a desirable locus for DNA barcoding should be standardized so that large databases of sequences for that locus can be developed. Most of the taxa of interest have loci that are sequencable without species-specific PCR primers. CBOL Plant Working Group, “A DNA barcode for land plants” PNAS 106(31):12794-12797 (2009). Further, these putative barcode loci are believed short enough to be easily sequenced with current technology. Kress et al., “DNA barcodes: Genes, genomics, and bioinformatics” PNAS 105(8):2761-2762 (2008). Consequently, these loci would provide a large variation between species in combination with a relatively small amount of variation within a species. Lahaye et al., “DNA barcoding the floras of biodiversity hotspots” Proc Natl Acad Sci USA 105(8):2923-2928 (2008).

DNA barcoding is based on a relatively simple concept. For example, most eukaryote cells contain mitochondria, and mitochondrial DNA (mtDNA) has a relatively fast mutation rate, which results in significant variation in mtDNA sequences between species and, in principle, a comparatively small variance within species. A 648-bp region of the mitochondrial cytochrome c oxidase subunit 1 (CO1) gene was proposed as a potential ‘barcode’. As of 2009, databases of CO1 sequences included at least 620,000 specimens from over 58,000 species of animals, larger than databases available for any other gene. Ausubel, J., “A botanical macroscope” Proceedings of the National Academy of Sciences 106(31):12569 (2009).

Software for DNA barcoding requires integration of a field information management system (FIMS), laboratory information management system (LIMS), sequence analysis tools, workflow tracking to connect field data and laboratory data, database submission tools and pipeline automation for scaling up to eco-system scale projects. Geneious Pro can be used for the sequence analysis components, and the two plugins made freely available through the Moorea Biocode Project, the Biocode LIMS and Genbank Submission plugins handle integration with the FIMS, the LIMS, workflow tracking and database submission.

Additionally, other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci US A. February 17; 106(7):2289-94).

In another embodiment, single cells are analyzed by digital polymerase chain reactions (PCR), e.g., Fluidigm C. The single cell data can then be correlated with the fluorescent signal determined using the fusion protein of the present invention. Digital polymerase chain reaction (digital PCR, DigitalPCR, dPCR, or dePCR) is a refinement of conventional polymerase chain reaction methods that can be used to directly quantify and clonally amplify nucleic acids including DNA, cDNA or RNA. The key difference between dPCR and traditional PCR lies in that PCR carries out one reaction per single sample and dPCR carries out a single reaction within samples separated into a large number of partitions wherein the reactions are carried out in each partition individually. A sample is partitioned so that individual nucleic acid molecules within the sample are localized and concentrated within many separate regions. The capture or isolation of individual nucleic acid molecules may be effected in micro well plates, capillaries, the dispersed phase of an emulsion, and arrays of miniaturized chambers, as well as on nucleic acid binding surfaces.

In a preferred embodiment, single cell analysis is performed using microfluidics. The single cell analysis is then correlated to the fluorescent signal of the fusion protein of the present invention. Microfluidics involves micro-scale devices that handle small volumes of fluids. Because microfluidics may accurately and reproducibly control and dispense small fluid volumes, in particular volumes less than 1 μl, application of microfluidics provides significant cost-savings. The use of microfluidics technology reduces cycle times, shortens time-to-results, and increases throughput. Furthermore, incorporation of microfluidics technology enhances system integration and automation. Microfluidic reactions are generally conducted in microdroplets. The ability to conduct reactions in microdroplets depends on being able to merge different sample fluids and different microdroplets. See, e.g., US Patent Publication No. 20120219947 and PCT publication No. WO2014085802 A1.

Droplet microfluidics offers significant advantages for performing high-throughput screens and sensitive assays. Droplets allow sample volumes to be significantly reduced, leading to concomitant reductions in cost. Manipulation and measurement at kilohertz speeds enable up to 108 samples to be screened in a single day. Compartmentalization in droplets increases assay sensitivity by increasing the effective concentration of rare species and decreasing the time required to reach detection thresholds. Droplet microfluidics combines these powerful features to enable currently inaccessible high-throughput screening applications, including single-cell and single-molecule assays. See, e.g., Guo et al., Lab Chip, 2012, 12, 2146-2155.

The manipulation of fluids to form fluid streams of desired configuration, discontinuous fluid streams, droplets, particles, dispersions, etc., for purposes of fluid delivery, product manufacture, analysis, and the like, is a relatively well-studied art. Microfluidic systems have been described in a variety of contexts, typically in the context of miniaturized laboratory (e.g., clinical) analysis. Other uses have been described as well. For example, WO 2001/89788; WO 2006/040551; U.S. Patent Application Publication No. 2009/0005254; WO 2006/040554; U.S. Patent Application Publication No. 2007/0184489; WO 2004/002627; U.S. Pat. No. 7,708,949; WO 2008/063227; U.S. Patent Application Publication No. 2008/0003142; WO 2004/091763; U.S. Patent Application Publication No. 2006/0163385; WO 2005/021151; U.S. Patent Application Publication No. 2007/0003442; WO 2006/096571; U.S. Patent Application Publication No. 2009/0131543; WO 2007/089541; U.S. Patent Application Publication No. 2007/0195127; WO 2007/081385; U.S. Patent Application Publication No. 2010/0137163; WO 2007/133710; U.S. Patent Application Publication No. 2008/0014589; U.S. Patent Application Publication No. 2014/0256595; and WO 2011/079176. In a preferred embodiment single cell analysis is performed in droplets using methods according to WO 2014085802. Each of these patents and publications is herein incorporated by reference in their entireties for all purposes.

Microfluidics may also be used to separate the single cells. Single cells can be separated using microfluidic devices based on the fluorescent signal from the fusion protein. Microfluidics involves micro-scale devices that handle small volumes of fluids. Because microfluidics may accurately and reproducibly control and dispense small fluid volumes, in particular volumes less than 1 μl, application of microfluidics provides significant cost-savings. The use of microfluidics technology reduces cycle times, shortens time-to-results, and increases throughput. The small volume of microfluidics technology improves amplification and construction of DNA libraries made from single cells and single isolated aggregations of cellular constituents. Furthermore, incorporation of microfluidics technology enhances system integration and automation.

Single cells of the present invention may be divided into single droplets using a microfluidic device. The single cells and/or single isolated aggregations of cellular constituents in such droplets may be further labeled with a barcode. In this regard reference is made to Macosko et al., 2015, “Highly Parallel Genome-wide Expression Profiling of Individual Cells Using Nanoliter Droplets” Cell 161, 1202-1214 and Klein et al., 2015, “Droplet Barcoding for Single-Cell Transcriptomics Applied to Embryonic Stem Cells” Cell 161, 1187-120,1 all the contents and disclosure of each of which are herein incorporated by reference in their entirety. Not being bound by a theory, the volume size of an aliquot within a droplet may be as small as 1 fL.

Single cells may be sorted into separate vessels by dilution of the sample and physical movement, such as pipetting. A machine can control the pipetting and separation. The machine may be a computer controlled robot. Any means of cell sorting may be used in the present invention. In Preferred embodiments, fluorescence-activated cell sorting (FACS) is used to sort cells based on the fluorescence signal derived from the fusion protein of the present invention. The cells may be sorted, such that individual cells are analyzed. In one embodiment, cells may be sorted into high fluorescence and low fluorescence groups. Each group may be analyzed for gene expression, protein expression, or perturbation constructs.

As used herein, “Adoptive cell transfer” (ACT) refers to the transfer of cells into a patient. The cells may have originated from the patient him or herself and then been altered before being transferred back, or, they may have come from another individual. T cells can be obtained from a number of sources, including peripheral blood mononuclear cells, bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. In one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. ln a further embodiment, the time period is at least 1, 2, 3, 4, 5, or 6 hours. In yet another preferred embodiment, the time period is 10 to 24 hours. ln one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such as in isolating tumor infiltrating lymphocytes (TIL) from tumor tissue or from immunocompromised individuals. Further, use of longer incubation times can increase the efficiency of capture of CD8+ T cells.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease as described herein. Antigen-specific cells for use in expansion may also be generated in vitro using any number of methods known in the art, for example, as described in U.S. Patent Publication No. US 20040224402 entitled, Generation And Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In one embodiment of the invention, the method further may comprise expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

Aspects of the invention involve the adoptive transfer of immune system cells, such as T cells, specific for selected antigens, such as tumor associated antigens (see Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; and, Restifo et al., 2015, Adoptive immunotherapy for cancer: harnessing the T cell response. Nat. Rev. Immunol. 12(4): 269-281). Various strategies may for example be employed to genetically modify T cells by altering the specificity of the T cell receptor (TCR) for example by introducing new TCR a and chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, U.S. Pat. No. 8,088,379). WO2013039889, WO2014018863, WO2014083175; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322). Alternative CAR constructs may be characterized as belonging to successive generations. First- generation CARs typically consist of a single-chain variable fragment of an antibody specific for an antigen, for example which may comprise a V_{L} linked to a V_{H} of a specific antibody, linked by a flexible linker, for example by a CD8α hinge domain and a CD8α transmembrane domain, to the transmembrane and intracellular signaling domains of either CD3ç or FcRγ (scFv-CD3ç or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ç; see U.S. Pat. Nos. 8,911,993; 8,916,381; 8,975,071; 9,101,584; 9,102,760; 9,102,761). Third-generation CARs include a combination of costimulatory endodomains, such a CD3ç-chain, CD97, GDI1a-CD18, CO2, ICOS, CD27, CD154, CDS, OX40, 4-1BB, or CD28 signaling domains (for example scFv-CD28-4-1BB-CD3ç or scFv-CD28-OX40-CD3ç; see U.S. Pat. Nos. 8,906,682; 8,399,645; 5,686,281; PCT Publication No. WO2014134165; PCT Publication No. WO2012079000). Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects.

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ç and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

Approaches such as the foregoing may be adapted to provide methods of treating and/or increasing survival of a subject having a disease, such as a neoplasia, for example by administering an effective amount of an immunoresponsive cell which may comprise an antigen recognizing receptor that binds a selected antigen, wherein the binding activates the immunoreponsive cell, thereby treating or preventing the disease (such as a neoplasia, a pathogen infection, an autoimmune disorder, or an allogeneic transplant reaction). Dosing in CAR T cell therapies may for example involve administration of from 10⁶ to 10⁹ cells/kg, with or without a course of lymphodepletion, for example with cyclophosphamide. Such dosing is also applicable to adoptive cell transfer of expanded T cells. Doses may be single doses of T cells or multiple doses. There is evidence from animal models (in nonlymphopenic hosts) suggesting that multiple doses of adoptively transferred T cells are superior to a single infusion of T cell/(June, C. H., Adoptive T cell therapy for cancer in the clinic. J Clin Invest. 2007 Jun. 1; 117(6): 1466-1476; and Kircher M. F., et al. In vivo high resolution three-dimensional imaging of antigen-specific cytotoxic T-lymphocyte trafficking to tumors. Cancer Res. 2003; 63:6838-6846).

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation. In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)). In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853).

In certain embodiments, the neoplasia may be selected from the group consisting of squamous cell cancer, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulva! cancer, thyroid cancer, hepatic carcinoma, anal carcinoma, penile carcinoma, head cancer and neck cancer.

Adoptive cell therapy may be administered in combination with other treatments. Incorporation of the therapy described herein may depend on a treatment step in the standard of care that causes the immune system to be suppressed. Such treatment steps may include irradiation, high doses of alkylating agents and/or methotrexate, steroids such as glucosteroids, surgery, such as to remove the lymph nodes, imatinib mesylate, high doses of TNF, and taxanes (Zitvogel et al., Immunological aspects of cancer chemotherapy. Nat Rev Immunol. 2008 January; 8(1):59-73). The T cell therapy may be administered before such steps or may be administered after. Advantageously, the treatment steps are administered as part of adoptive T-cell therapy.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. App. Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); European Patents EP 2 784 162 B1 and EP 2 771 468 B1; European Patent Applications EP 2 771 468 (EP13818570.7), EP 2 764 103 (EP13824232.6), and EP 2 784 162 (EP14170383.5); and PCT Patent Publications PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/07 4812), WO 2014/093622 (PCT/US2013/074667), WO 2014/093635 (PCT/US2013/07 4691), WO 2014/093655 (PCT/US2013/074736), WO 2014/093712 (PCT/US2013/074819), WO2014/093701 (PCT/US2013/074800), WO2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809). Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/836,127, 61/836,101, 61/836,080 and 61/835,973, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT Patent applications Nos: PCT/US2014/041803, PCT/US2014/041800, PCT/US2014/041809, PCT/US2014/041804 and PCT/US2014/041806, each filed Jun. 10, 2014 6/10/14; PCT/US2014/041808 filed Jun. 11, 2014; and PCT/US2014/62558 filed Oct. 28, 2014, and U.S. Provisional Patent Applications Ser. Nos.: 61/915,150, 61/915,301, 61/915,267 and 61/915,260, each filed Dec. 12, 2013; 61/757,972 and 61/768,959, filed on Jan. 29, 2013 and Feb. 25, 2013; 61/835,936, 61/836,127, 61/836,101, 61/836,080, 61/835,973, and 61/835,931, filed Jun. 17, 2013; 62/010,888 and 62/010,879, both filed Jun. 11, 2014; 62/010,329 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/054,490, 62/055,484, 62/055,460 and 62/055,487, each filed Sep. 25, 2014; and 62/069,243, filed Oct. 27, 2014. Reference is also made to U.S. provisional patent applications Nos. 62/055,484, 62/055,460, and 62/055,487, filed Sep. 25, 2014; U.S. provisional patent application 61/980,012, filed Apr. 15, 2014; and U.S. provisional patent application 61/939,242 filed Feb. 12, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013. Reference is made to U.S. provisional patent application U.S. Ser. No. 61/980,012 filed Apr. 15, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to U.S. provisional patent applications 61/915,251; 61/915,260 and 61/915,267, each filed on Dec. 12, 2013.

Mention is also made of U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. application 62/091,462, 12 Dec. 2014, DEAD GUIDES FOR CRISPR TRANSCRIPTION FACTORS; U.S. application 62/096,324, 23 Dec. 2014, DEAD GUIDES FOR CRISPR TRANSCRIPTION FACTORS; U.S. application 62/091,456, 12 Dec. 2014, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, RNA-TARGETING SYSTEM; U.S. application 62/096,656, 24 Dec. 2014, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; US application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/054,675, 24 Sep. 2014, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Each of these patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

Also with respect to general information on CRISPR-Cas Systems, mention is made of the following (also hereby incorporated herein by reference):
Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L A. Nat Biotechnol March; 31(3):233-9 (2013);
One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013);
Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013);
Genome engineering using the CRISPR-Cas9 system. Ran, F A., Hsu, P D., Wright, J., Agarwala, V., Scott, D A., Zhang, F. Nature Protocols November; 8(11):2281-308 (2013-B);
Genome-Scale CRISPR-Cas9 Knockout Screening in Human Cells. Shalem, O. Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print];
Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. April 20. doi: 10.1038/nbt.2889 (2014);
CRISPR-Cas9 Knockin Mice for Genome Editing and Cancer Modeling. Platt R J, Chen S, Zhou Y, Yim M J, Swiech L, Kempton H R, Dahlman J E, Parnas O, Eisenhaure T M, Jovanovic M, Graham D B, Jhunjhunwala S, Heidenreich M, Xavier R J, Langer R, Anderson D G, Hacohen N, Regev A, Feng G, Sharp P A, Zhang F. Cell 159(2): 440-455 DOI: 10.1016/j.cell.2014.09.014(2014);
Development and Applications of CRISPR-Cas9 for Genome Engineering, Hsu P D, Lander E S, Zhang F., Cell. June 5; 157(6):1262-78 (2014).
Genetic screens in human cells using the CRISPR/Cas9 system, Wang T, Wei J J, Sabatini D M, Lander E S., Science. January 3; 343(6166): 80-84. doi:10.1 126/science. 1246981 (2014);
Rational design of highly active sgRNAs for CRISPR-Cas9-mediated gene inactivation, Doench J G, Hartenian E, Graham D B, Tothova Z, Hegde M, Smith I, Sullender M, Ebert B L, Xavier R J, Root D E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M D, Trevino A E, Joung J, Abudayyeh O O, Barcena C, Hsu P D, Habib N, Gootenberg J S, Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N E, Zheng K, Shalem O, Lee K, Shi X, Scott D A, Song J, Pan J Q, Weissleder R, Lee H, Zhang F, Sharp P A. Cell 160, 1246-1260, Mar. 12, 2015 (multiplex screen in mouse), and
In vivo genome editing using Staphylococcus aureus Cas9, Ran F A, Cong L, Yan W X, Scott D A, Gootenberg J S, Kriz A J, Zetsche B, Shalem O, Wu X, Makarova K S, Koonin E V, Sharp P A, Zhang F., (published online 1 Apr. 2015), Nature. April 9; 520(7546):186-91 (2015).
Shalem et al., “High-throughput functional genomics using CRISPR-Cas9,” Nature Reviews Genetics I 6, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., “CRISPR/Cas9 cleavage of viral DNA efficiently suppresses hepatitis B virus,” Scientific Reports 5:10833. doi: 10.1038/srep10833 (Jun. 2, 2015)
Nishimasu et al., “Crystal Structure of Staphylococcus aureus Cas9,” Cell 162, 1113-1126 (Aug. 27, 2015)
Zetsche et al., “Cpfl Is a Single RNA-Guided Endonuclease of a Class 2 CRISPR-Cas System,” Cell 163, 1-13 (Oct. 22, 2015)
Shmakov et al., “Discovery and Functional Characterization of Diverse Class 2 CRISPR-Cas Systems,” Molecular Cell 60, 1-13 (Available online Oct. 22, 2015)

each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single-and multinucleotide changes carried on editing templates. The study showed that simultaneous use of two crRNAs enabled multiplex mutagenesis. Furthermore, when the approach was used in combination with recombineering, in S. pneumoniae, nearly I 00% of cells that were recovered using the described approach contained the desired mutation, and in E. coli, 65% that were recovered contained the mutation.
Wang et al. (2013) used the CRISPR/Cas system for the one-step generation of mice carrying mutations in multiple genes which were traditionally generated in multiple steps by sequential recombination in embryonic stem cells and/or time-consuming intercrossing of mice with a single mutation. The CRISPR/Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately offset guide RNAs is required for double-stranded breaks and extends the number of specifically recognized bases for target cleavage. The authors demonstrated that using paired nicking can reduce off-target activity by 50-to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and sgRNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 m complex with sgRNA and its target DNA at 2.5 AO resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays. Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR/Cas9 knockout and nucleotide preference at the cleavage site. The authors also found that the sequence preference for CRISPRi/a is substantially different from that for CRISPR/Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.
Zetsche et al. (2015) reported the characterization of Cpfl, a putative class 2 CRISPR effector. It was demonstrated that Cpfl mediates robust DNA interference with features distinct from Cas9. Identifying this mechanism of interference broadens our understanding of CRISPR-Cas systems and advances their genome editing applications.
Shmakov et al. (2015) reported the characterization of three distinct Class 2 CRISPR-Cas systems. The effectors of two of the identified systems, C2c1 and C2c3, contain RuvC like endonuclease domains distantly related to Cpfl. The third system, C2c2, contains an effector with two predicted HEPN RNase domains.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

In some embodiments, one or more functional domains are associated with the CRISPR enzyme, for example a Type II Cas9 enzyme.

In some embodiments, one or more functional domains are associated with an adaptor protein, for example as used with the modified guides of Konnerman et al. (Nature 517, 583-588, 29 Jan. 2015).

In some embodiments, one or more functional domains are associated with an dead sgRNA (dRNA). In some embodiments, a dRNA complex with active cas9 directs gene regulation by a functional domain at on gene locus while an sgRNA directs DNA cleavage by the active cas9 at another locus, for example as described by Dahlman et al., ‘Orthogonal gene control with a catalytically active Cas9 nuclease’ (in press). In some embodiments, dRNAs are selected to maximize selectivity of regulation for a gene locus of interest compared to off-target regulation. In some embodiments, dRNAs are selected to maximize target gene regulation and minimize target cleavage

For the purposes of the following discussion, reference to a functional domain could be a functional domain associated with the CRISPR enzyme or a functional domain associated with the adaptor protein.

In some embodiments, the one or more functional domains is an NLS (Nuclear Localization Sequence) or an NES (Nuclear Export Signal). In some embodiments, the one or more functional domains is a transcriptional activation domain may comprise VP64, p65, MyoD1, HSF1, RTA, SET7/9 and a histone acetyltransferase. Other references herein to activation (or activator) domains in respect of those associated with the CRISPR enzyme include any known transcriptional activation domain and specifically VP64, p65, MyoD1, HSF1, RTA, SET7/9 or a histone acetyltransferase.

In some embodiments, the one or more functional domains is a transcriptional repressor domain. In some embodiments, the transcriptional repressor domain is a KRAB domain. In some embodiments, the transcriptional repressor domain is a NuE domain, NcoR domain, SID domain or a SID4X domain.

In some embodiments, the one or more functional domains have one or more activities which may comprise methylase activity, demethylase activity, transcription activation activity, transcription repression activity, transcription release factor activity, histone modification activity, RNA cleavage activity, DNA cleavage activity, DNA integration activity or nucleic acid binding activity.

Histone modifying domains are also preferred in some embodiments. Exemplary histone modifying domains are discussed below. Transposase domains, HR (Homologous Recombination) machinery domains, recombinase domains, and/or integrase domains are also preferred as the present functional domains. In some embodiments, DNA integration activity includes HR machinery domains, integrase domains, recombinase domains and/or transposase domains. Histone acetyltransferases are preferred in some embodiments.

In some embodiments, the DNA cleavage activity is due to a nuclease. In some embodiments, the nuclease may comprise a FokI nuclease.

In some embodiments, the one or more functional domains is attached to the CRISPR enzyme so that upon binding to the sgRNA and target the functional domain is in a spatial orientation allowing for the functional domain to function in its attributed function.

In some embodiments, the one or more functional domains is attached to the adaptor protein so that upon binding of the CRISPR enzyme to the sgRNA and target, the functional domain is in a spatial orientation allowing for the functional domain to function in its attributed function.

In an aspect the invention provides a composition as herein discussed wherein the one or more functional domains is attached to the CRISPR enzyme or adaptor protein via a linker, optionally a GlySer linker, as discussed herein.

Endogenous transcriptional repression is often mediated by chromatin modifying enzymes such as histone methyltransferases (HMTs) and deacetylases (HDACs). Repressive histone effector domains are known and an exemplary list is provided below. In the exemplary table, preference was given to proteins and functional truncations of small size to facilitate efficient viral packaging (for instance via AAV). In general, however, the domains may include HDACs, histone methyltransferases (HMTs), and histone acetyltransferase (HAT) inhibitors, as well as HDAC and HMT recruiting proteins. The functional domain may be or include, in some embodiments, HDAC Effector Domains, HDAC Recruiter Effector Domains, Histone Methyltransferase (HMT) Effector Domains, Hi stone Methyltransferase (HMT) Recruiter Effector Domains, or Histone Acetyltransferase Inhibitor Effector Domains.

HDAC Effector Domains

| | | | | | Full | Selected | Final | |
|---|---|---|---|---|---|---|---|---|
| Subtype/ | | Substrate | Modification | | size | truncation | size | Catalytic |
| Complex | Name | (if known) | (if known) | Organism | (aa) | (aa) | (aa) | domain |
| HDAC I | HDAC8 | — | — | X laevis | 325 | 1-325 | 325 | 1-272: |
| | | | | | | | | HDAC |
| HDAC I | RPD3 | — | — | S. cerevisiae | 433 | 19-340 | 322 | 19-331: |
| | | | | | | | (Vannier) | HDAC |
| HDAC IV | MesoLo4 | — | — | M loti | 300 | 1-300 | 300 | — |
| | | | | | | (Gregoretti) | | |
| HDAC IV | HDAC1 1 | — | — | H. sapiens | 347 | 1-347 (Gao) | 347 | 14-326: |
| | | | | | | | | HDAC |
| HD2 | HDTI | — | — | A. thaliana | 245 | 1-211 (Wu) | 211 | — |
| SIRT I | SIRT3 | H3K9Ac | — | H. sapiens | 399 | 143-399 | 257 | 126-382: |
| | | H4K16Ac | | | | (Scher) | | SIRT |
| | | H3K56Ac | | | | | | |
| SIRTI | HST2 | — | — | C. albicans | 331 | 1-331 | 331 | — |
| | | | | | | (Hnisz) | | |
| SIRTI | CobB | — | — | E. coli | 242 | 1-242 | 242 | — |
| | | | | (K12) | | (Landry) | | |
| SIRT I | HST2 | — | — | S. cerevisiae | 357 | 8-298 | 291 | — |
| | | | | | | (Wilson) | | |
| SIRT III | SIRT5 | H4K8Ac | — | H. sapiens | 310 | 37-310 | 274 | 41-309: |
| | | H4K16Ac | | | | (Gertz) | | SIRT |
| SIRT III | Sir2A | — | — | P. falciparum | 273 | 1-273 (Zhu) | 273 | 19-273: |
| | | | | | | | | SIRT |
| SJRT IV | SIRT6 | H3K9Ac | — | H. sapiens | 355 | 1-289 | 289 | 35-274: |
| | | H3K56Ac | | | | (Tennen) | | SIRT |

Accordingly, the repressor domains of the present invention may be selected from histone methyltransferases (HMTs), histone deacetylases (HDACs), histone acetyltransferase (HAT) inhibitors, as well as HDAC and HMT recruiting proteins.

The HDAC domain may be any of those in the table above, namely: HDAC8, RPD3, MesoLo4, HDAC1 1, HDT1, SIRT3, HST2, CobB, HST2, SIRT5, Sir2A, or SIRT6.

In some embodiment, the functional domain may be a HDAC Recruiter Effector Domain. Preferred examples include those in the Table below, namely MeCP2, MBD2b, Sin3a, NcoR, SALLI, RCOR1. NcoR is exemplified in the present Examples and, although preferred, it is envisaged that others in the class will also be useful.

Table of HDAC Recruiter Effector Domains

| | | | | | Full | Selected | Final | |
|---|---|---|---|---|---|---|---|---|
| Subtype/ | | Substrate | Modification | | size | truncation | size | Catalytic |
| Complex | Name | (if known) | (if known) | Organism | (aa) | (aa) | (aa) | domain |
| Sin3a | MeCP2 | — | — | R. norvegicus | 492 | 207-492 | 286 | — |
| Sin3a | MBD2b | — | — | H. sapiens | 262 | 45-262 | 218 | — |
| | | | | | | (Boeke) | | |
| Sin3a | Sin3a | — | — | H. sapiens | 1273 | 524-851 | 328 | 627-829: |
| | | | | | | (Laherty) | | HDAC |
| | | | | | | | | interaction |
| NcoR | NcoR | — | — | H. sapiens | 2440 | 420-488 | 69 | — |
| | | | | | | (Zhang) | | |
| NuRD | SALLI | — | — | M musculus | 1322 | 1-93 | 93 | — |
| | | | | | | (Lauberth) | | |
| CoREST | RCORI | H3K9Ac | — | H. sapiens | 482 | 81-300 (Gu, | 220 | — |
| | | H4K16Ac | | | | Ouyang) | | |
| | | H3K56Ac | | | | | | |

In some embodiment, the functional domain may be a Methyltransferase (HMT) Effector Domain. Preferred examples include those in the Table below, namely NUE, vSET, EHMT2/G9A, SUV39H1, dim-5, KYP, SUVR4, SET4, SETI, SETD8, and TgSET8. NUE is exemplified in the present Examples and, although preferred, it is envisaged that others in the class will also be useful.

Table of Histone Methyltransferase (HMT) Effector Domains

| | | | | | Full | Selected | Final | |
|---|---|---|---|---|---|---|---|---|
| Subtype/ | | Substrate | Modification | | size | truncation | size | Catalytic |
| Complex | Name | (if known) | (if known) | Organism | (aa) | (aa) | (aa) | domain |
| SET | NUE | H2B, | — | C. trachomatis | 219 | 1-219 | 219 | — |
| | | H3, H4 | | | | (Pennini) | | |
| SET | vSET | — | H3K27me3 | P. bursaria | 119 | 1-119 | 119 | 4-112: |
| | | | | chlorella | | (Mujtaba) | | SET2 |
| | | | | virus | | | | |
| SUV39 | EHMT2/G9 A | Hl.4K2, | H3K9mel/2, | M musculus | 1263 | 969-1263 | 295 | 1025-1233: |
| family | | H3K9, | H1K25mel | | | (Tachibana) | | preSET, |
| | | H3K27 | | | | | | SET, |
| | | | | | | | | postSET |
| SUV39 | SUV39Hl | — | H3K9me2/3 | H. sapiens | 412 | 79-412 | 334 | 172-412: |
| | | | | | | (Snowden) | | preSET, |
| | | | | | | | | SET, |
| | | | | | | | | postSET |
| Suvar3-9 | dim-5 | — | H3K9me3 | N. crassa | 331 | 1-331 | 331 | 77-331: |
| | | | | | | (Rathert) | | preSET, |
| | | | | | | | | SET, |
| | | | | | | | | postSET |
| Suvar3-9 | KYP | — | H3K9mel/2 | A. thaliana | 624 | 335-601 | 267 | — |
| (SUVH | | | | | | | (Jackson) | |
| subfamily) | | | | | | | | |
| Suvar3-9 | SUVR4 | H3K9me 1 | H3K9me2/3 | A. thaliana | 492 | 180-492 | 313 | 192-462: |
| (SUVR | | | | | | | (Thorstensen) | preSET, |
| subfamily) | | | | | | | | SET, |
| | | | | | | | | postSET |
| Suvar4-20 | SET4 | — | H4K20me3 | C. elegans | 288 | 1-288 | 288 | — |
| | | | | | | (Vielle) | | |
| SET8 | SETI | — | H4K20mel | C. elegans | 242 | 1-242 | 242 | — |
| | | | | | | (Vielle) | | |
| SET8 | SETD8 | — | H4K20mel | H. sapiens | 393 | 185-393 | 209 | 256-382: |
| | | | | | | | (Couture) | SET |
| SET8 | TgSET8 | — | H4K20mel/2/3 | T gondii | 1893 | 1590-1893 | 304 | 1749-1884: |
| | | | | | | (Sautel) | | SET |

In some embodiment, the functional domain may be a Histone Methyltransferase (HMT) Recruiter Effector Domain. Preferred examples include those in the Table below, namely Hp1a, PHF19, and NIPP1.

Table of Histone Methyltransferase (HMT) Recruiter Effector Domains

| | | | | | Full | Selected | Final | |
|---|---|---|---|---|---|---|---|---|
| Subtype/ | | Substrate | Modification | | size | truncation | size | Catalytic |
| Complex | Name | (if known) | (if known) | Organism | (aa) | (aa) | (aa) | domain |
| — | Hpla | — | H3K9me3 | M musculus | 191 | 73-191 | 119 | 121-179: |
| | | | | | | | (Hathaway) | chromoshadow |
| — | PHF19 | — | H3K27me3 | H. sapiens | 580 | (1-250) + | 335 | 163-250: PHD2 |
| | | | | | | GGSG | (Ballaé) | |
| | | | | | | linker+ | | |
| | | | | | | (500-580) | | |
| — | NIPPI | — | H3K27me3 | H. sapiens | 351 | 1-329 (Jin) | 329 | 310-329: EED |

In some embodiment, the functional domain may be Histone Acetyltransferase Inhibitor Effector Domain. Preferred examples include SET/TAF-1p listed in the Table below.

Table of Histone Acetyltransferase Inhibitor Effector Domains

| | | | | | Full | Selected | Final | |
|---|---|---|---|---|---|---|---|---|
| Subtype/ | | Substrate | Modification | | size | truncation | size | Catalytic |
| Complex | Name | (if known) | (if known) | Organism | (aa) | (aa) | (aa) | domain |
| — | SET/TAF-1β | — | — | M musculus | 289 | 1-289 | 289 | — |
| | | | | | | (Cervoni) | | |

It is also preferred to target endogenous (regulatory) control elements (such as enhancers and silencers) in addition to a promoter or promoter-proximal elements. Thus, the invention can also be used to target endogenous control elements (including enhancers and silencers) in addition to targeting of the promoter. These control elements can be located upstream and downstream of the transcriptional start site (TSS), starting from 200 bp from the TSS to 100 kb away. Targeting of known control elements can be used to activate or repress the gene of interest. In some cases, a single control element can influence the transcription of multiple target genes. Targeting of a single control element could therefore be used to control the transcription of multiple genes simultaneously.

Targeting of putative control elements on the other hand (e.g. by tiling the region of the putative control element as well as 200 bp up to 100 kB around the element) can be used as a means to verify such elements (by measuring the transcription of the gene of interest) or to detect novel control elements (e.g. by tiling 100 kb upstream and downstream of the TSS of the gene of interest). In addition, targeting of putative control elements can be useful in the context of understanding genetic causes of disease. Many mutations and common SNP variants associated with disease phenotypes are located outside coding regions. Targeting of such regions with either the activation or repression systems described herein can be followed by readout of transcription of either a) a set of putative targets (e.g. a set of genes located in closest proximity to the control element) orb) whole-transcriptome readout by e.g. RNAseq or microarray. This would allow for the identification of likely candidate genes involved in the disease phenotype. Such candidate genes could be useful as novel drug targets.

Histone acetyltransferase (HAT) inhibitors are mentioned herein. However, an alternative in some embodiments is for the one or more functional domains to comprise an acetyltransferase, preferably a histone acetyltransferase. These are useful in the field of epigenomics, for example in methods of interrogating the epigenome. Methods of interrogating the epigenome may include, for example, targeting epigenomic sequences. Targeting epigenomic sequences may include the guide being directed to an epigenomic target sequence. Epigenomic target sequence may include, in some embodiments, include a promoter, silencer or an enhancer sequence.

Use of a functional domain linked to a CRISPR-Cas enzyme as described herein, preferably a dead-Cas, more preferably a dead-Cas9, to target epigenomic sequences can be used to activate or repress promoters, silencer or enhancers.

Examples of acetyltransferases are known but may include, in some embodiments, histone acetyltransferases. In some embodiments, the histone acetyltransferase may comprise the catalytic core of the human acetyltransferase p300 (Gerbasch & Reddy, Nature Biotech 6 Apr. 2015).

In some preferred embodiments, the functional domain is linked to a dead-Cas9 enzyme to target and activate epigenomic sequences such as promoters or enhancers. One or more guides directed to such promoters or enhancers may also be provided to direct the binding of the CRISPR enzyme to such promoters or enhancers.

The term “associated with” is used here in relation to the association of the functional domain to the CRISPR enzyme or the adaptor protein. It is used in respect of how one molecule ‘associates’ with respect to another, for example between an adaptor protein and a functional domain, or between the CRISPR enzyme and a functional domain. In the case of such protein-protein interactions, this association may be viewed in terms of recognition in the way an antibody recognizes an epitope. Alternatively, one protein may be associated with another protein via a fusion of the two, for instance one subunit being fused to another subunit. Fusion typically occurs by addition of the amino acid sequence of one to that of the other, for instance via splicing together of the nucleotide sequences that encode each protein or subunit. Alternatively, this may essentially be viewed as binding between two molecules or direct linkage, such as a fusion protein. In any event, the fusion protein may include a linker between the two subunits of interest (i.e. between the enzyme and the functional domain or between the adaptor protein and the functional domain). Thus, in some embodiments, the CRISPR enzyme or adaptor protein is associated with a functional domain by binding thereto. In other embodiments, the CRISPR enzyme or adaptor protein is associated with a functional domain because the two are fused together, optionally via an intermediate linker.

Attachment of a functional domain or fusion protein can be via a linker, e.g., a flexible glycine-serine (GlyGlyGlySer) (SEQ ID NO: 1) or (GGGS)₃ (SEQ ID NO: 2) or a rigid alpha-helical linker such as (Ala(GluAlaAlaAlaLys)Ala) (SEQ ID NO: 3). Linkers such as (GGGGS)₃ (SEQ ID NO: 4) are preferably used herein to separate protein or peptide domains. (GGGGS)₃ (SEQ ID NO: 4) is preferable because it is a relatively long linker (15 amino acids). The glycine residues are the most flexible and the serine residues enhance the chance that the linker is on the outside of the protein. (GGGGS)₆ (SEQ ID NO: 5), (GGGGS)₉ (SEQ ID NO: 6) or (GGGGS)₁₂ (SEQ ID NO: 7) may preferably be used as alternatives. Other preferred alternatives are (GGGGS)₁ (SEQ ID NO: 8), (GGGGS)₂ (SEQ ID NO: 9), (GGGGS)₄ (SEQ ID NO: 10), (GGGGS)₅ (SEQ ID NO: 11), (GGGGS)₇ (SEQ ID NO: 12), (GGGGS)₈ (SEQ ID NO: 13), (GGGGS)₁₀ (SEQ ID NO: 14), or (GGGGS)₁₁ (SEQ ID NO: 15). Alternative linkers are available, but highly flexible linkers are thought to work best to allow for maximum opportunity for the 2 parts of the Cas9 to come together and thus reconstitute Cas9 activity. One alternative is that the NLS of nucleoplasmin can be used as a linker. For example, a linker can also be used between the Cas9 and any functional domain. Again, a (GGGGS)₃ (SEQ ID NO: 4) linker may be used here (or the 6, 9, or 12 repeat versions therefore) or the NLS of nucleoplasmin can be used as a linker between Cas9 and the functional domain.

With respect to use of the CRISPR-Cas system generally, mention is made of the documents, including patent applications, patents, and patent publications cited throughout this disclosure as embodiments of the invention can be used as in those documents. CRISPR-Cas System(s) can be used to perform efficient and cost effective functional genomic screens. Such screens can utilize CRISPR-Cas genome wide libraries. Such screens and libraries can provide for determining the function of genes, cellular pathways genes are involved in, and how any alteration in gene expression can result in a particular biological process. An advantage of the present invention is that the CRISPR system avoids off-target binding and its resulting side effects. This is achieved using systems arranged to have a high degree of sequence specificity for the target DNA.

A genome wide library may comprise a plurality of CRISPR-Cas system guide RNAs, as described herein, which may comprise guide sequences that are capable of targeting a plurality of target sequences in a plurality of genomic loci in a population of eukaryotic cells. The population of cells may be a population of embryonic stem (ES) cells. The target sequence in the genomic locus may be a non-coding sequence. The non-coding sequence may be an intron, regulatory sequence, splice site, 3′ UTR, 5′ UTR, or polyadenylation signal. Gene function of one or more gene products may be altered by said targeting. The targeting may result in a knockout of gene function. The targeting of a gene product may comprise more than one guide RNA. A gene product may be targeted by 2, 3, 4, 5, 6, 7, 8, 9, or 10 guide RNAs, preferably 3 to 4 per gene. Off-target modifications may be minimized (See, e.g., DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013)), incorporated herein by reference. The targeting may be of about 100 or more sequences. The targeting may be of about 1000 or more sequences. The targeting may be of about 20,000 or more sequences. The targeting may be of the entire genome. The targeting may be of a panel of target sequences focused on a relevant or desirable pathway. The pathway may be an immune pathway. The pathway may be a cell division pathway.

One aspect of the invention comprehends a genome wide library that may comprise a plurality of CRISPR-Cas system guide RNAs that may comprise guide sequences that are capable of targeting a plurality of target sequences in a plurality of genomic loci, wherein said targeting results in a knockout of gene function. This library may potentially comprise guide RNAs that target each and every gene in the genome of an organism.

In some embodiments of the invention the organism or subject is a eukaryote (including mammal including human) or a non-human eukaryote or a non-human animal or a non-human mammal. In some embodiments, the organism or subject is a non-human animal, and may be an arthropod, for example, an insect, or may be a nematode. In some methods of the invention the organism or subject is a plant. In some methods of the invention the organism or subject is a mammal or a non-human mammal. A non-human mammal may be for example a rodent (preferably a mouse or a rat), an ungulate, or a primate. In some methods of the invention the organism or subject is algae, including microalgae, or is a fungus.

The knockout of gene function may comprise: introducing into each cell in the population of cells a vector system of one or more vectors which may comprise an engineered, non-naturally occurring CRISPR-Cas system which may comprise I. a Cas protein, and II. one or more guide RNAs, wherein components I and II may be same or on different vectors of the system, integrating components I and II into each cell, wherein the guide sequence targets a unique gene in each cell, wherein the Cas protein is operably linked to a regulatory element, wherein when transcribed, the guide RNA which may comprise the guide sequence directs sequence-specific binding of a CRISPR-Cas system to a target sequence in the genomic loci of the unique gene, inducing cleavage of the genomic loci by the Cas protein, and confirming different knockout mutations in a plurality of unique genes in each cell of the population of cells thereby generating a gene knockout cell library. The invention comprehends that the population of cells is a population of eukaryotic cells, and in a preferred embodiment, the population of cells is a population of embryonic stem (ES) cells.

The one or more vectors may be plasmid vectors. The vector may be a single vector which may comprise Cas9, a sgRNA, and optionally, a selection marker into target cells. Not being bound by a theory, the ability to simultaneously deliver Cas9 and sgRNA through a single vector enables application to any cell type of interest, without the need to first generate cell lines that express Cas9. The regulatory element may be an inducible promoter. The inducible promoter may be a doxycycline inducible promoter. In some methods of the invention the expression of the guide sequence is under the control of the T7 promoter and is driven by the expression of T7 polymerase. The confirming of different knockout mutations may be by whole exome sequencing. The knockout mutation may be achieved in 100 or more unique genes. The knockout mutation may be achieved in 1000 or more unique genes. The knockout mutation may be achieved in 20,000 or more unique genes. The knockout mutation may be achieved in the entire genome. The knockout of gene function may be achieved in a plurality of unique genes which function in a particular physiological pathway or condition. The pathway or condition may be an immune pathway or condition. The pathway or condition may be a cell division pathway or condition.

The invention also provides kits that comprise the genome wide libraries mentioned herein. The kit may comprise a single container which may comprise vectors or plasmids which may comprise the library of the invention. The kit may also comprise a panel which may comprise a selection of unique CRISPR-Cas system guide RNAs which may comprise guide sequences from the library of the invention, wherein the selection is indicative of a particular physiological condition. The invention comprehends that the targeting is of about 100 or more sequences, about 1000 or more sequences or about 20,000 or more sequences or the entire genome. Furthermore, a panel of target sequences may be focused on a relevant or desirable pathway, such as an immune pathway or cell division.

In an additional aspect of the invention, a Cas9 enzyme may comprise one or more mutations and may be used as a generic DNA binding protein with or without fusion to a functional domain. The mutations may be artificially introduced mutations or gain-or loss-of-function mutations. The mutations may include but are not limited to mutations in one of the catalytic domains (D10 and H840) in the RuvC and HNH catalytic domains, respectively. Further mutations have been characterized. In one aspect of the invention, the functional domain may be a transcriptional activation domain, which may be VP64. In other aspects of the invention, the functional domain may be a transcriptional repressor domain, which may be KRAB or SID4X. Other aspects of the invention relate to the mutated Cas 9 enzyme being fused to domains which include but are not limited to a transcriptional activator, repressor, a recombinase, a transposase, a histone remodeler, a demethylase, a DNA methyltransferase, a cryptochrome, a light inducible/controllable domain or a chemically inducible/controllable domain. Some methods of the invention can include inducing expression of targeted genes. In one embodiment, inducing expression by targeting a plurality of target sequences in a plurality of genomic loci in a population of eukaryotic cells is by use of a functional domain.

Useful in the practice of the instant invention, reference is made to:
Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science December 12. (2013). [Epub ahead of print]; Published in final edited form as: Science. 2014 Jan. 3; 343(6166): 84-87.
Shalem et al. involves a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1 . The authors observed a high level of consistency between independent guide RNAs targeting the same gene and a high rate of hit confirmation, and thus demonstrated the promise of genome-scale screening with Cas9.

Reference is also made to US patent publication number US20140357530; and PCT Patent Publication WO2014093701, hereby incorporated herein by reference.

With respect to use of the CRISPR-Cas system generally, mention is made of the documents, including patent applications, patents, and patent publications cited throughout this disclosure as embodiments of the invention can be used as in those documents. CRISPR-Cas System(s) can be used to perform saturating or deep scanning mutagenesis of genomic loci in conjunction with a cellular phenotype-for instance, for determining critical minimal features and discrete vulnerabilities of functional elements required for gene expression, drug resistance, and reversal of disease. By saturating or deep scanning mutagenesis is meant that every or essentially every DNA base is cut within the genomic loci. A library of CRISPR-Cas guide RNAs may be introduced into a population of cells. The library may be introduced, such that each cell receives a single guide RNA (sgRNA). In the case where the library is introduced by transduction of a viral vector, as described herein, a low multiplicity of infection (MOI) is used. The library may include sgRNAs targeting every sequence upstream of a (protospacer adjacent motif) (PAM) sequence in a genomic locus. The library may include at least 100 non-overlapping genomic sequences upstream of a PAM sequence for every 1000 base pairs within the genomic locus. The library may include sgRNAs targeting sequences upstream of at least one different PAM sequence. The CRISPR-Cas System(s) may include more than one Cas protein. Any Cas protein as described herein, including orthologues or engineered Cas proteins that recognize different PAM sequences may be used. The frequency of off target sites for a sgRNA may be less than 500. Off target scores may be generated to select sgRNAs with the lowest off target sites. Any phenotype determined to be associated with cutting at a sgRNA target site may be confirmed by using sgRNA's targeting the same site in a single experiment. Validation of a target site may also be performed by using a nickase Cas9, as described herein, and two sgRNAs targeting the genomic site of interest. Not being bound by a theory, a target site is a true hit if the change in phenotype is observed in validation experiments.

The genomic loci may include at least one continuous genomic region. The at least one continuous genomic region may comprise up to the entire genome. The at least one continuous genomic region may comprise a functional element of the genome. The functional element may be within a non-coding region, coding gene, intronic region, promoter, or enhancer. The at least one continuous genomic region may comprise at least I kb, preferably at least 50 kb of genomic DNA. The at least one continuous genomic region may comprise a transcription factor binding site. The at least one continuous genomic region may comprise a region of DNase I hypersensitivity. The at least one continuous genomic region may comprise a transcription enhancer or repressor element. The at least one continuous genomic region may comprise a site enriched for an epigenetic signature. The at least one continuous genomic DNA region may comprise an epigenetic insulator. The at least one continuous genomic region may comprise two or more continuous genomic regions that physically interact. Genomic regions that interact may be determined by ‘4C technology’. 4C technology allows the screening of the entire genome in an unbiased manner for DNA segments that physically interact with a DNA fragment of choice, as is described in Zhao et al. ((2006) Nat Genet 38, 1341-7) and in U.S. Pat. No. 8,642,295, both incorporated herein by reference in its entirety. The epigenetic signature may be histone acetylation, histone methylation, histone ubiquitination, histone phosphorylation, DNA methylation, or a lack thereof.

CRISPR-Cas System(s) for saturating or deep scanning mutagenesis can be used in a population of cells. The CRISPR-Cas System(s) can be used in eukaryotic cells, including but not limited to mammalian and plant cells. The population of cells may be prokaryotic cells. The population of eukaryotic cells may be a population of embryonic stem (ES) cells, neuronal cells, epithelial cells, immune cells, endocrine cells, muscle cells, erythrocytes, lymphocytes, plant cells, or yeast cells.

In one aspect, the present invention provides for a method of screening for functional elements associated with a change in a phenotype. In preferred embodiments, elements involved in secretion are screened for. The library may be introduced into a population of cells that are adapted to contain a Cas protein. The cells may be sorted into at least two groups based on the phenotype. The phenotype may be expression of a gene, cell growth, cell viability, or secretion. The relative representation of the guide RNAs present in each group are determined, whereby genomic sites associated with the change in phenotype are determined by the representation of guide RNAs present in each group. The change in phenotype may be a change in expression of a gene of interest. The gene of interest may be upregulated, downregulated, or knocked out. The cells may be sorted into a high expression group and a low expression group. The population of cells may include a reporter construct that is used to determine the phenotype. The reporter construct may include a detectable marker. Cells may be sorted by use of the detectable marker. The detectable marker may be a labeled fusion protein of the present invention.

Useful in the practice of the instant invention, reference is made to the article entitled BCL11A enhancer dissection by Cas9-mediated in situ saturating mutagenesis. Canver, M.C., Smith, E. C., Sher, F., Pinello, L., Sanjana, N. E., Shalem, O., Chen, D. D., Schupp, P. G., Vinjamur, D. S., Garcia, S. P., Luc, S., Kurita, R., Nakamura, Y., Fujiwara, Y., Maeda, T., Yuan, G., Zhang, F., Orkin, S. H., & Bauer, D. E. DOI:10.1038/nature15521, published online Sep. 16, 2015, the article is herein incorporated by reference and discussed briefly below: Canver et al. describes novel pooled CRISPR-Cas9 guide RNA libraries to perform in situ saturating mutagenesis of the human and mouse BCL11A erythroid enhancers previously identified as an enhancer associated with fetal hemoglobin (HbF) level and whose mouse ortholog is necessary for erythroid BCL11A expression. This approach revealed critical minimal features and discrete vulnerabilities of these enhancers. Through editing of primary human progenitors and mouse transgenesis, the authors validated the BCL11A erythroid enhancer as a target for HbF reinduction. The authors generated a detailed enhancer map that informs therapeutic genome editing.

Through this disclosure and the knowledge in the art, CRISPR-Cas system, or components thereof or nucleic acid molecules thereof (including, for instance HDR template) or nucleic acid molecules encoding or providing components thereof may be delivered by a delivery system herein described both generally and in detail.

Vector delivery, e.g., plasmid, viral delivery: The CRISPR enzyme, for instance a Cas9, and/or any of the present RNAs, for instance a guide RNA, can be delivered using any suitable vector, e.g., plasmid or viral vectors, such as adeno associated virus (AAV), lentivirus, adenovirus or other viral vector types, or combinations thereof. Cas9 and one or more guide RNAs can be packaged into one or more vectors, e.g., plasmid or viral vectors.

Regarding type 2 diabetes (T2D) associated regions of the genome reference is made to the following:

Diabetes Genetics Initiative Genome-wide association analysis identifies loci for type 2 diabetes and triglyceride levels. Science. 2007; 316:1331-1336.

Scott L J, et al. A genome-wide association study of type 2 diabetes in Finns detects multiple susceptibility variants. Science. 2007; 316: 1341-1345.

Wellcome Trust Case Control Consortium Genome-wide association study of 14,000 cases of seven common diseases and 3,000 shared controls. Nature. 2007; 447:661-678.

Zeggini E, et al. Replication of genome-wide association signals in UK samples reveals risk loci for type 2 diabetes. Science. 2007; 316:1336-1341.

Steinthorsdottir V, et al. A variant in CDKAL1 influences insulin response and risk of type 2 diabetes. Nat. Genet. 2007; 39:770-775.

Sladek R, et al. A genome-wide association study identifies novel risk loci for type 2 diabetes. Nature. 2007; 445:881-885.

Florez J C, et al. A 1O0K genome-wide association scan for diabetes and related traits in the Framingham Heart Study: replication and integration with other genome-wide datasets. Diabetes. 2007; 56:3063-3074.

Rampersaud E, et al. Identification of novel candidate genes for type 2 diabetes from a genome-wide association scan in the Old Order Amish: evidence for replication from diabetes-related quantitative traits and from independent populations. Diabetes. 2007; 56:3053-3062.

Hanson R L, et al. A search for variants associated with young-onset type 2 diabetes in American Indians in a 100K genotyping array. Diabetes. 2007; 56:3045-52.

Hayes M G, et al. Identification of type 2 diabetes genes in Mexican Americans through genome-wide association studies. Diabetes. 2007; 56:3033-3044.

Salonen J, et al. Type 2 diabetes whole-genome association study in four populations: the DiaGen consortium. Am. J. Hum. Genet. 2007; 81:338-345.

Zeggini E, et al. Meta-analysis of genome-wide association data and large-scale replication identifies additional susceptibility loci for type 2 diabetes. Nat Genet. 2008 May; 40(5):638-45. doi: 10.1038/ng.120. Epub 2008 Mar. 30.

The present invention advantageously provides for high-throughput, pooled assays of the regulated secretion of diverse biologically-active molecules, including hormones, neurotransmitters, cytokines, chemokines, enzymes, and growth factors. The reporter can be used to screen genes and compounds regulating secretion in specific cellular contexts, eliminating the need for expensive, laborious and technically challenging immunoassays to directly measure secreted molecules. Moreover, because each cell can be interrogated and isolated based on its response to stimulation (using fluorescence-activated cell sorting), the system is well-suited to pooled screens, including CRISPR nuclease, CRISPR transactivator, and MITE-Seq approaches.

The present invention advantageously provides for comprehensive interrogation of genetic functional elements or genes for roles in secretion using pooled screening strategies. The present invention provides the ability to scale directly from single gene investigation to genome wide screen, without additional assay development. The present invention provides for the first time a universal regulated secretion assay that does not require the creation of a new assay for each secreted substance of interest and no need for expensive, laborious, technically challenging immunoassays.

As such, the present invention may be applied to explore therapeutic targets in: metabolic disease, neuronal disorders, psychiatric disease, inflammatory and autoimmune disorders, cancer and metastasis, senescence and aging, cardiovascular disease, infectious disease, and other conditions in which secreted proteins play a pathophysiologic or therapeutic role.

The present invention also provides for the ability to sort cells based on levels of secretion. This is especially useful in sorting T cells used for adoptive cell transfer therapies.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### SynaptoSNAP System

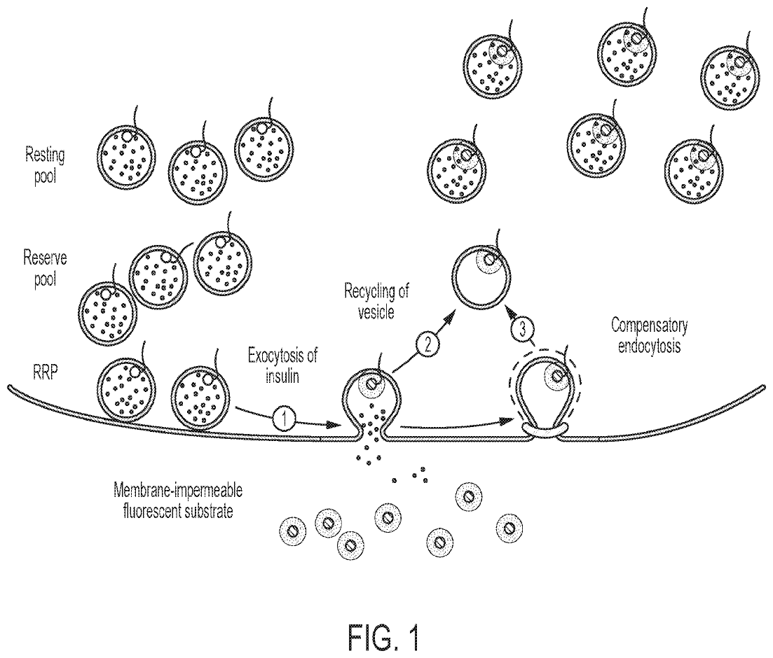

Turning to FIG. 1, the regulated secretion of molecules can be measured in a cell-autonomous manner by tracking the flux of secretory vesicles to the cell membrane. The amount of a substance secreted through the regulated pathway is directly proportional to, and highly correlated with, the number of substance-containing vesicles that fuse with the cell membrane during exocytosis. By tracking the flow of vesicles to the cell membrane, Applicants can closely approximate the secretion of substances carried by those vesicles.

Applicants designed a system to accurately track the flux of vesicles using a tagged version of a protein in the synaptic vesicle membrane, synaptobrevin (a.k.a., vesicle-associated membrane protein-2, or VAMP2). The C-terminus of this transmembrane protein resides within the lumen of the vesicles, and becomes exposed to the extracellular environment when the vesicle fuses to the cell membrane during exocytosis. Applicants added a commercially available “SNAP tag” (New England Biolabs) to the C-terminus of synaptobrevin, which allows for the protein to be irreversibly labeled with a cell-impermeable, fluorescent chemical substrate, under standard cell culture conditions. The SNAP tag is a 20 kDa mutant of the DNA repair protein 06-alkylguanine-DNA alkyltransferase (AGT) that reacts specifically and rapidly with benzyl guanine derivatives, leading to irreversible covalent labeling of the SNAP-tag with a synthetic probe. (See, e.g., www.neb.com/tools-and-resources/feature-articles/snap-tag-technologies-novel-tools-to-study -protein-function; Lancet. 2013 Apr. 20; 381(9875):1371-9; and Nucleic Acids Res. 2014 August; 42(14): el 12).

At rest, cells expressing the SNAP-tagged version of synaptobrevin (“synaptoSNAP”) are non-fluorescent, with the protein residing primarily within secretory vesicles. Upon stimulation, the protein travels with the vesicles to the cell surface, gets exposed to the extracellular environment during exocytosis, and covalently binds to the cell-impermeable, fluorescent (“SNAP Surface”) substrate that added to the media. Through vesicle recycling and compensatory endocytosis, the now-fluorescent synaptoSNAP protein is incorporated into newly-formed, intracellular vesicles, contributing to an accumulation of fluorescence within the cells as secretion proceeds (FIG. 1). Fluorescence intensity of the cells serves as a close proxy for secretion of the substance of interest.

### Example 2

### SynaptoSNAP Detects Secretion in Response to a Stimulus

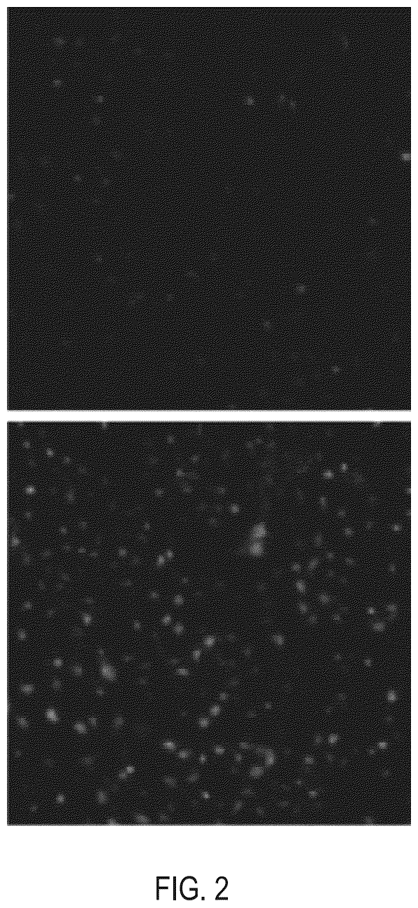

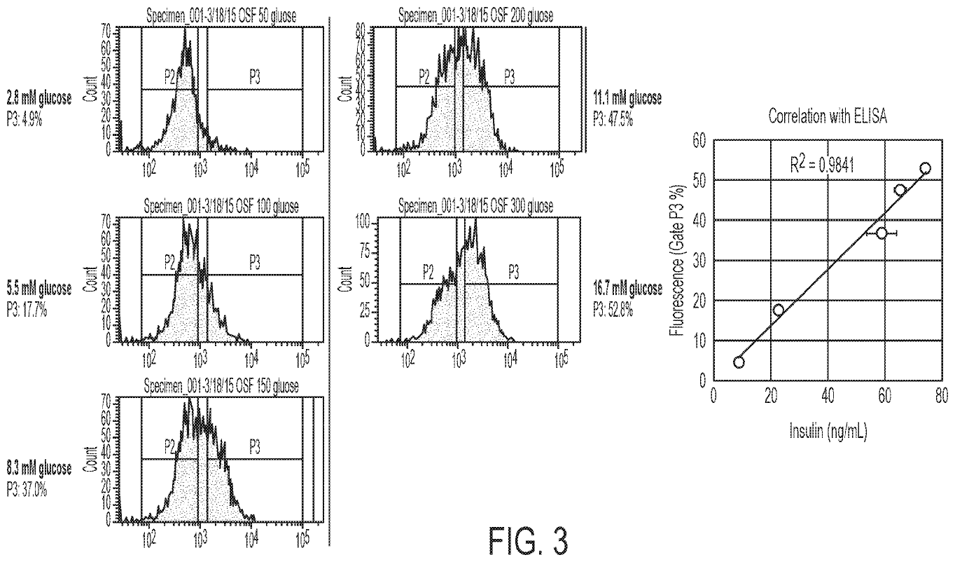

Turning to FIGS. 2, 3 and 4, Applicants have used this reporter in the context of pancreatic beta cells to track insulin secretion in response to various stimuli. Applicants expressed synaptoSNAP in the INS-1E rat beta-cell line and show that fluorescence increases upon exposure to high glucose (FIG. 2). Moreover, the intensity of the fluorescence rises in proportion to the strength of stimulation, and in close correlation with insulin secretion, as measured by ELISA (Mercodia rat insulin ELISA) (FIG. 3). The intensity of cellular fluorescence is proportional to the degree of glucose stimulation and correlates well with the amount of insulin secreted as detected with an insulin ELISA.

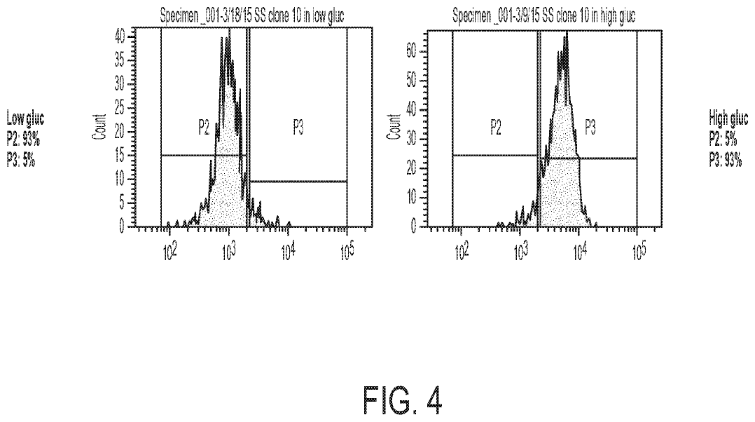

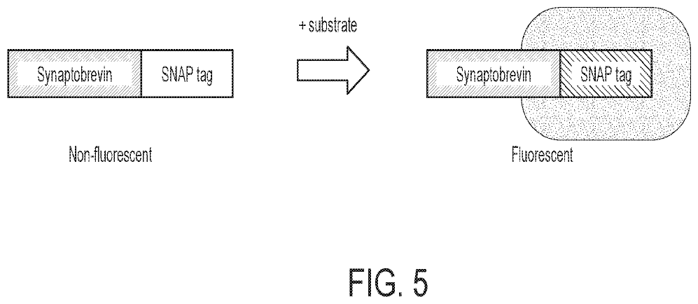

Cells expressing the reporter eventually recover to their original non-fluorescent state, enabling the isolation of clones with improved assay characteristics, including greater change in fluorescence upon stimulation (FIG. 4). Shown is FACS analysis for clone 10 in low glucose and high glucose conditions.

### Example 3

### Determining Genes Involved in the Secretion

The present invention may be used to determine genes or genetic elements involved in secretion. As an example, Applicants created a beta-cell line that expresses the reporter. The cell line can be interrogated with a genome-wide CRISPR nuclease library targeting the genes that are expressed in the beta cell by introducing the CRISPR library into the cells as described herein. Additionally, saturating mutagenesis libraries targeting functional DNA sequences, such as promoters, enhancers and repressors may be used. Genome-wide libraries targeting every gene in a genome may also be used. Such libraries are described herein. Genes discovered to regulate secretion of bioactive molecules represent potentially valuable drug targets. Pooled screens allow for high-throughput assessment of genes nominated by human genetics in disease pathophysiology. The library may contain guide RNAs that can be identified upon sequencing. Guide RNAs may be expressed from vectors each including a unique barcode sequence. As an example, beta cells are stimulated with glucose or another secretagogue. The cells are sorted by flow cytometry into low and high secretors, based on the fluorescence intensity of each single cell. Next generation sequencing is performed to detect enrichment of CRISPR guides in each sorted population, and thereby genes are determined that play a role in secretion. This exemplary approach could be used to determine the genes involved in the regulated secretion of any biomolecule or secretion from any cell type.

### Example 4

### Comprehensively Evaluate the Significance to Neuronal Function of Every Possible Coding Variant in CACNA1C

CACNA1C is a calcium channel gene that is linked to five psychiatric diseases (i.e., autism, ADHD, bipolar, depression, schizophrenia) (Lancet. 2013 Apr. 20; 381(9875):1371-9). CACNA1C encodes an alpha-I subunit of a voltage-dependent calcium channel. Calcium channels mediate the influx of calcium ions into the cell upon membrane polarization. The alpha-1 subunit consists of 24 transmembrane segments and forms the pore through which ions pass into the cell. The calcium channel consists of a complex of alpha-I, alpha-2/delta, beta, and gamma subunits in a 1:1:1:1 ratio. There are multiple isoforms of each of these proteins, either encoded by different genes or the result of alternative splicing of transcripts. The protein encoded by this gene binds to and is inhibited by dihydropyridine. Alternative splicing results in many transcript variants encoding different proteins. Some of the predicted proteins may not produce functional ion channel subunits.

In this example a neuronal cell line expressing the reporter is created. The CACNA1C gene is knocked out using transient expression of CRISPR/Cas9. A MITE-Seq library (Mutagenesis by Integrated TilEs) is created encoding every possible coding variant of CACNA1C in a suitable expression vector (Nucleic Acids Res. 2014 August; 42(14):el 12). The MITE-Seq library is introduced into the cells. The cells are stimulated for secretion by depolarizing the cells. The cells are sorted by flow cytometry into low and high secretors based on their fluorescence intensity. This exemplary approach could be used to determine the effect of genetic variation in any gene that plays a role in the regulated secretion of bioactive molecules. Coding variants identified as impacting protein function will enable genetic risk assessment, and may inform the personalization of therapies.

A comprehensive assessment of variant impact will enable rapid interpretation of exome sequencing data using “look up” tables containing functional annotation of every possible coding variant.

Applicants are also using the cell line to perform a comprehensive investigation of coding variants in HNF1A, a known diabetes gene with important roles in beta-cell function.

### Example 5

Treatment with Anti-Tumor T Cells

Lymphocytes are collected from a patient by leukopheresis. The reporter is introduced into the pool of lymphocytes. The T cells are stimulated to secrete IFN-y using tumor- specific antigens (e.g., CEA, HER2), or if unknown, samples derived from the patient's cancer. The cells are sorted by Flow to isolate tumor-reactive cells based on their high fluorescence. The T-cell receptors on these anti-tumor immune cells are characterized by sequencing. Single cell sequencing may be used. Specific targets in the tumor may be determined. The patient lymphocytes with these anti-tumor receptors are expanded or CAR-T cells are engineered to express these specific receptors. The T cells are then reintroduced into the patient as a personalized medical therapy. Not being bound by a theory, the present invention allows the detection of secretion in pooled T cell populations that are then sorted, thus greatly improving the efficiency of screening anti-tumor T cells. Previously, individual T cell clones would need to be isolated either for single cell sequencing or for cytokine secretion assays. Identification of the patient's own T cells with anti-tumor activity could enable a highly focused, cell-based immunotherapy to eradicate only the cancer cells, with minimal off-target toxicity.

### Example 6

### Alternative Applications

The present invention may be used in any application where secretion is determined, such as, but not limited to hormone secretion, e.g., insulin, ghrelin, PPy, or somatostatin, macrophage secretion of cytokines, TLR-signaling, matrix metalloproteinase (MMP) activity for cancer, stem cell differentiation, senescence secretion, and exosome secretion and trafficking. The present invention may be used for sorting any cells based on secretion.

Regarding Type 2 diabetes, the present invention has broad applicability for many secretory systems and phenotypes. The present invention may be used for pooled screens for insulin secretion phenotypes in rodent and human pancreatic beta cells. This approach can be used in conjunction screening technologies that include creating genetic mutations using large scale CRISPR technologies to perturb thousands of genes implicated in pathogenesis of type 2 diabetes. In initial studies, Applicants measure insulin secretion from pancreatic beta cells, however there are numerous secreted hormones that impact type 2 diabetes etiology, such as glucagon from alpha cells, somatostatin from delta cells, pancreatic polypeptide from PP cells, and ghrelin from epsilon cells.

Regarding cancer, the present invention has multiple applications in cancer models. Cancer invasion and metastasis include the secretion of proteases for the extracellular matrix including matrix metalloproteinases. These genes have long been known to have great potential as therapeutic targets for preventing cancer metastasis, however current MMP inhibitors have shown high-toxicity and low specificity for inhibiting the MMPs which drive metastasis. (Rakashanda et al., Role of proteases in cancer: A Review, 2012, Biotechnology and Molecular Biology Review Vol. 7(4), 90-101). Cells are stimulated to secrete MMP in conjunction with the perturbation of genes to determine target genes for inhibition of MMP secretion. One method known in the art to stimulate secretion of MMP is the treatment of cells with conditioned media from stromal fibroblasts. Not being bound by a theory, fibroblasts secrete several growth factors, such as SCF, TGF-β, HGF and IGF that can stimulate secretion of MMP.

Regarding inflammation, cytokine secretion is involved in a large number of processes, including carcinogenesis, response to pathogen infection, inflammation, and immune response to tumor formation. The synaptoSNAP technology has great applicability for exploring immune responses to each stimuli. (cytokines in cancer pathogenesis and cancer therapy, Glenn Danoff, Nature Reviews, 2004).

Regarding senescence and aging, the biological process of aging of organisms and tissues precisely correlates with the cellular process of senescence. Senescence is a permanent removal from the cell cycle of a cell in response to DNA damage, and cell stress. Senescent cells no longer function within the tissue of which they are apart. During aging as tissues accumulate senescent cells, organ function declines contributing to age related diseases such as cancer, diabetes and heart disease. Senescent cells secrete pro-inflammatory factors which further contribute to decline in organ function with age. At present there are no measurable indicators of cell age and further there are no therapeutics that abrogate the deleterious effects of the accumulation of senescent cells in an organ. However, expression of the gene CDKN2A is known to strongly correlate with senescence response across many tissue types.

The synaptoSNAP technology can be driven from the CDKN2A promoter to enable the measurement of senescence-associated secretion both in cell models and in vivo animal models. This enables genetic and chemical screens to discover the factors involved in senescence associated secretion and provides a method for therapeutic screens to abrogate the effects of aging. Furthermore, synaptoSNAP as a direct readout of senescence associated secretion can serve as an immediately measurable marker of biological age of a cell and tissue, which is an invaluable tool for the study of mechanisms of both tissue and whole organism rates of aging.

Regarding the tracking of exosome secretion, while the synaptoSNAP fluorescent secretion reporter was designed to track exocytosis of cargo from large vesicles, it also can be used to detect the release of exosomes, which are small, membrane-bound vesicles that carry biologically active molecules, including mRNA, miRNA, proteins and lipids, from one cell to another distal cell. All cells appear to secrete these intercellular messengers, either constitutively or upon cell type-specific stimulation. There is intense and growing interest in using exosomes for diagnostic and therapeutic applications, yet their small size (50-150 nm diameter) makes them challenging to isolate and characterize. In particular, reliable separation of exosomes from other similarly-sized vesicles that are released directly from the plasma membrane, or from lysed cells, remains impossible with current technologies. There are no known antigens specific to exosomes that can be used to pull down just these vesicles.

However, exosomes contain synaptobrevin-2. Therefore, the synaptoSNAP reporter also tracks to these small vesicles when introduced into cells. Exosomes could be isolated from a cell expressing synaptoSNAP, while excluding vesicles derived directly from the cell membrane via budding or lysis. First, the synaptoSNAP protein resident on the cell's surface can be blocked using a cell impermeable substrate (Surface-Block from NEB). Next, exosomes can be isolated by specific pulldown of the synaptoSNAP protein and the cargo of the exosomes can be analyzed for their RNA, DNA and protein contents. This technique could be combined with transgenic mouse models described herein to isolate exosomes within the circulation from specific tissues of origin, which has not been possible to date.

Regarding exosomes and cancer, the role exosome secretion plays in cancer metastasis is an area of active study. The synaptoSNAP technology could be used to track exosomes in vivo from the primary tumor to the tissues within an organism in which they traffic. This would have great applicability in not only enabling the tracking of exosomes from tumors but also accessing the contribution cancer-derived exosomes play in cancer progression.

### Example 7

### High-Throughput Insulin (INS) Secretion Screen in Rat Pancreatic Beta Cells (INS1E) Using the Synapto-SNAP Technology.

Applicants use the synaptoSNAP reporter in the context of a pancreatic beta-cell line to perform a CRISPR nuclease and a CRISPR transactivator screen. Applicants focused on genes implicated by human genetics in the pathogenesis of type 2 diabetes, but the system is not limited to such libraries. Applicants queried 6468 sgRNAs targeting 1078 genes in proximity to type 2 diabetes (T2D) associated regions of the genome. Applicants calculated an enrichment score for each gene for both increased and loss of INS secretion. The enrichment score is determined by plotting counts of each sgRNA in the increase or loss of INS secretion fraction against the relative abundance of each sgRNA in the initial library. The screen identified 151 genes that when knocked out cause a loss of INS secretion and identified 106 genes that when knocked out cause an increase in INS secretion. 152 genes displayed a significant >2 fold enrichment in the loss INS secretion fraction, and 106 genes displayed a significant >2 fold enrichment in the increase INS secretion fraction.

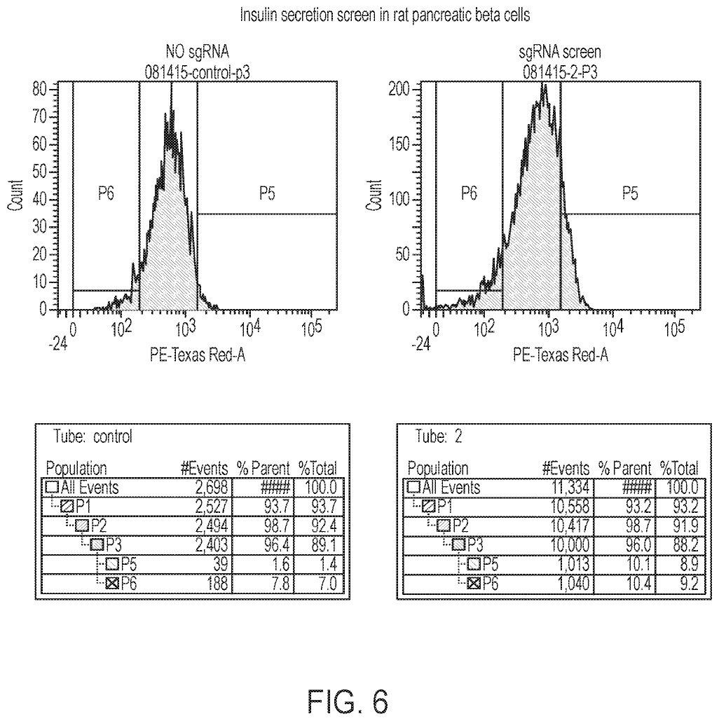

Turning to FIG. 6, shown are the results of 4×106 INS1E cells sorted by flow cytometry, treated with glucose, in the presence and absence of sgRNAs. Depicted is the fluorescence distribution of INS1E rat pancreatic beta cells in high glucose without any sgRNA/CRISPR-Cas9 treatment (left) and in the presence of 6468 sgRNAs/CRISPR-Cas9 targeting 1078 genes. The lower I 0% (P6) from the sgRNA treated are the loss of INS secretion cells. The upper 10% fraction (P5) from the sgRNA treated cells are the increased INS secretion cells.

These cells were infected with lenti-viral CRISPR libraries at a multiplicity of infection (MOI) of <0.3 viroids per cell, ensuring single copy integration. Cells were selected for 14 days in puromycin to ensure near complete gene knockout. Following selection, 4 million cells were treated with 1 6.7 mM glucose and analyzed for accumulation of Synapto-snap fluorescence (Texas Red). CRISPR library treatment resulted in an increase in the number of cells in both the loss and gain of secretion fractions as compared to untreated controls suggesting a screen dependent change in the distribution of INS secretion.

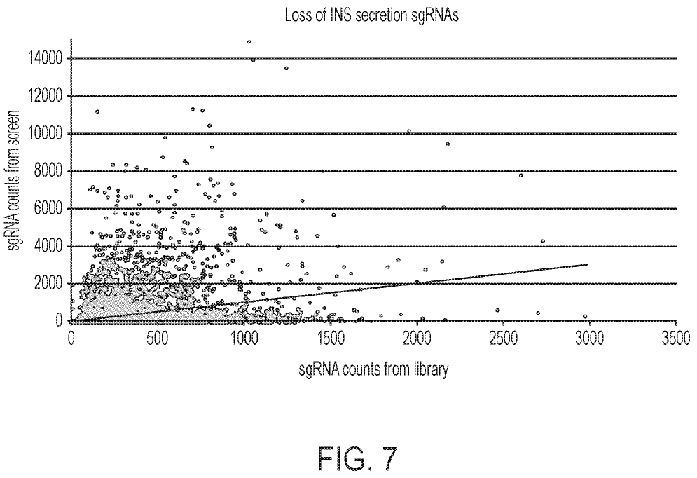

Turning to FIG. 7, genomic DNA from the loss of INS secretion fraction were sequenced by miseq and each sgRNA count was tallied. Counts of each sgRNA in the loss of INS secretion fraction is plotted against their relative abundance in the initial library. The black line represents the expected counts relative to library. SgRNAs above the line show a higher representation in the loss of INS secretion fraction than expected.

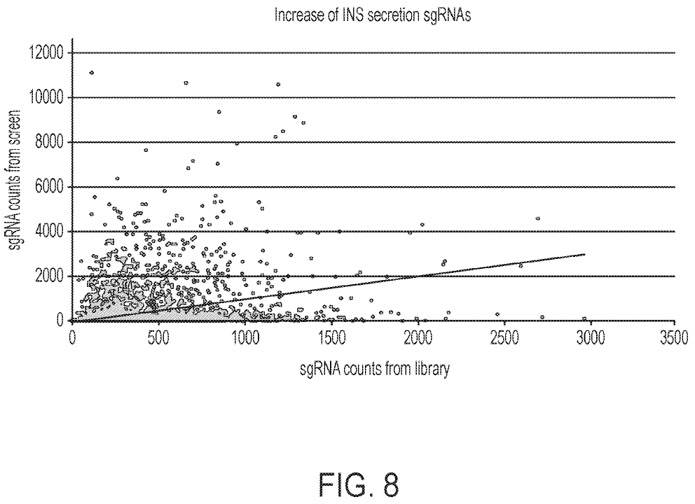

Turning to FIG. 8, genomic DNA from the increase of INS secretion fraction were sequenced by miseq and each sgRNA count was tallied. Counts of each sgRNA in the increase of INS secretion fraction is plotted against their relative abundance in the initial library. The black line represents the expected counts relative to library. SgRNAs above the line show a higher representation in the increase of INS secretion fraction than expected

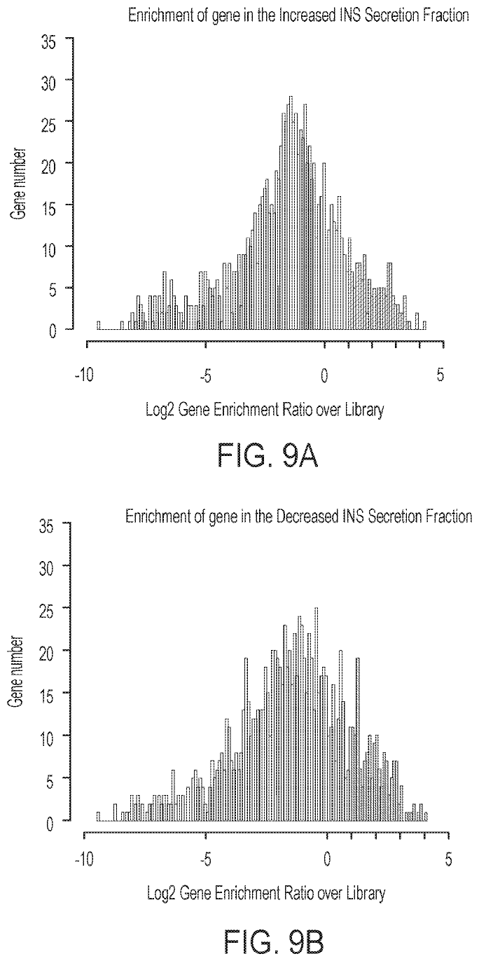

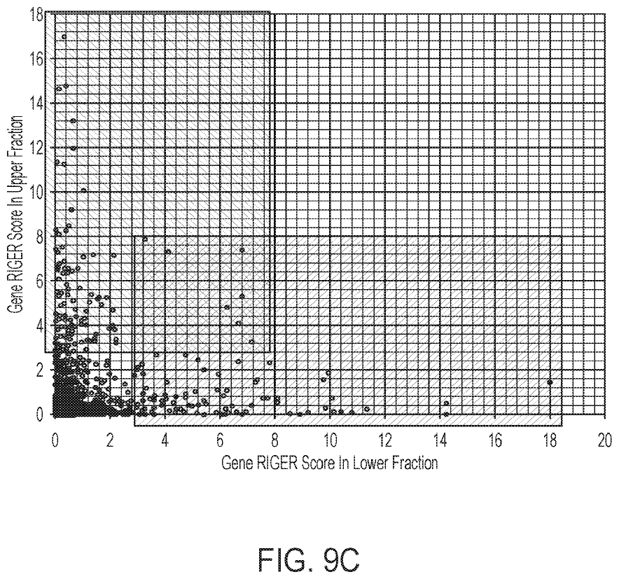

Turning to FIG. 9, measurements of multiple sgRNAs for each gene were collapsed into a single gene score by RIGER, which ranks sgRNAs according to their differential effects between two classes of samples, then identifies the genes targeted by the sgRNAs at the top of the list. In this way, RIGER identifies genes essential to the difference between the classes. For details, see Luo, et al. Proc Natl Acad Sci U S A. 2008, 105(51):20380-20385. Genes that displayed a consistent 2 fold enrichment score across sgRNA RIGER scores for each fraction were identified as candidate INS secretion genes (FIGS. 9A,B). The majority of genes identified in each fraction are specific to either increase or loss of INS secretion with 7/1078 genes enriched in both fractions (FIG. 9C).

### Protocols

### Glucose-Stimulated Fluorescence (Synaptosnap) Protocol

1. Aspirate growth medium, then add KRB with 50 mg/dL (2.8 mM) glucose, incubate 30 min to rest the cells.
2. Aspirate, then add KRB with 50 mg/dL (2.8 mM) glucose+4 uM Surface-Block (I: 1000 of 4 mM stock), incubate 60 min to block synaptoSNAP on the cell surface.
3. Aspirate, then add KRB with 2 uM Surface-549 (1:500 of 1 mM stock)+glucose and/or compound, incubate 6 hours to stimulate cells and acquire fluorescence.
4. Aspirate, then fix cells for flow analysis.
   Surface-Block: 50 uL of DMSO+200 nmol tube=4 mM stock. Store at −20C.
   Surface-549: 50 uL of DMSO+50 nmol tube=1 mM stock. Store at −20C.
   Krebs-Ringer bicarbonate buffer (KRB) (129 mm NaCl, 4.7 mm KC!, 1.2 mm KH2PO4, 5 mm NaHCO3, 10 mm HEPES, 3 mm d-glucose, 2.5 mm CaCl₂, 1.2 mm MgCl₂, and 0.2% BSA; pH adjusted to 7.4 with NaOH)

### INS1E Secretion Screen

Library Generation with 2 Bio-Reps:
1) Infect (−6×10⁷)×2 INS1E cells to achieve (3×107)×2 cells after selection (Starting population adjusted based on viral titer).
2) Infect 1×10⁶ Synato-snap INS1E cells with empty CRISPR virus for passaging
3) After 4-5 days from infection collect 15-30×10⁶ cells for early time point measurement (Freeze cell pellet).
4) Expand cells to 6×10⁷, at passage collect 15-30×10⁶ cells for a mid time point measurement (Freeze cell pellet).
5) Expand cells to 6×10⁷, at passage collect 15-30×10⁶ cells for a second-mid time point measurement (Freeze cell pellet).
6) Expand cells to 6×10⁷, collect 15-30×10⁶ for final time point, measurement (Freeze cell pellet).
   INS Secretion Screen with 2 Bio-Reps:
7) Collect 1×10⁷ Synato-snap INS1E cells infected with empty CRISPR and Synato-snap INS1E cells from each library bio-rep.
   a. Aspirate growth medium, then add KRB with 50 mg/dL (2.8 mM) glucose, incubate 30 min to rest the cells.
   b. Aspirate, then add KRB with 50 mg/dL (2.8 mM) glucose+4 uM Surface-Block (1:1000 of 4 mM stock), incubate 60 min to block synaptoSNAP on the cell surface.
   c. Aspirate, then add KRB with 2 uM Surface-549 (1:500 of 1 mM stock)+glucose and/or compound, incubate 6 hours to stimulate cells and acquire fluorescence.
   d. Aspirate, then fix cells for flow analysis.
1) Run 1×107 virus control cells through flow cytometer to obtain secretion distribution.
2) Cell sort I×107 Synato-snap INS IE cells from each library bio-rep.
   a. Collect 1×106 cells from the upper 10% fractions and the lower 10% fractions from each bio-rep.

| | | Collect | Collect | Collect | Collect | Flow | Sort High | Sort Low |
|---|---|---|---|---|---|---|---|---|
| | Infect | ETP | MTP_l | MTP_2 | FTP | Analysis | Fraction | Fraction |
| Control | 1 × 10⁶ | — | — | — | — | 1 × 10⁷ | — | — |
| Library 1 | 6 × 10⁷ | 3 × 10⁶ | 3 × 10⁶ | 3 × 10⁶ | 3 × 10⁶ | 1 × 10⁷ | 1 × 10⁶ | 1 × 10⁶ |
| Library 2 | 6 × 10⁷ | 3 × 10⁶ | 3 × 10⁶ | 3 × 10⁶ | 3 × 10⁶ | 1 × 10⁷ | 6 × 10⁶ | 1 × 10⁶ |
| Day | 0 | 4-5 | 8-9 | 12-13 | 16-17 | — | — | — |

Surface-Block: 50 uL of DMSO+200 nmol tube=4 mM stock. Store at −20C.
Surface-549: 50 uL of DMSO+50 nmol tube=1 mM stock. Store at −20C.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1-39. (canceled)

40. A method for measuring secretion from a single cell, the method comprising:
a) introducing a non-naturally occurring nucleic acid construct encoding a fusion protein to the single cell, wherein the encoded fusion protein: (i) is capable of being expressed in the single cell; (ii) is a vesicle membrane protein which has at least 90% identity to the amino acid sequence of VAMP1, VAMP2, VAMP3, VAMP4, VAMP5, VAMP7, VAMP8, synaptophysin or a synaptotagmin family protein, wherein the vesicle membrane protein comprises a cytoplasmic domain, a transmembrane domain and a vesicular domain, wherein the vesicular domain comprises a SNAP tag; and (iii) localizes to the membrane of a secretory vesicle such that the SNAP tag localizes to the lumen of the secretory vesicle, wherein the SNAP tag is capable of binding to a cell-impermeable marker upon secretion of the secretory vesicle;
b) exposing the single cell to the cell-impermeable marker; and
c) measuring endocytic uptake and accumulation of the cell-impermeable marker inside the single cell as a quantitative proxy for secretion from the single cell,
thereby measuring secretion from the single cell.

41. The method of claim 40, wherein the cell-impermeable marker is a fluorescent marker, optionally wherein the fluorescent marker is SNAP surface substrate.

42. The method of claim 41, wherein measurement of the accumulation of fluorescence in the single cells quantitates secretory vesicle-mediated secretion from the single cell.

43. The method of claim 40, wherein the cell-impermeable marker is introduced to extracellular media of the single cell.

44. The method of claim 40, wherein the single cell is a cell of an animal model, optionally wherein the animal model is a mouse.

45. The method of claim 44, wherein the cell-impermeable marker is injected into the animal model.

46. The method of claim 40, wherein the nucleic acid construct further comprises a regulatory sequence operably linked to the nucleic acid construct encoding the fusion protein.

47. The method of claim 40, wherein the nucleic acid construct further comprises a selective marker operably linked to a second regulatory sequence, optionally wherein the selective marker is an antibiotic resistance gene.

48. The method of claim 40, wherein the single cell is an endocrine cell, an exocrine cell, an immune cell, a hematopoietic cell, a neuron, a hepatocyte, a myocyte, a kidney cell, an adipocyte, an osteocyte, a stem cell or a cell line derived therefrom.

49. The method of claim 48, wherein the endocrine cell is a beta cell, an alpha cell, an L cell, a K cell, or a cell line derived therefrom.

50. The method of claim 48, wherein the immune cell is a B cell, a T cell, a CAR T cell, a natural killer cell, a monocyte, a macrophage, a plasma cell, a dendritic cell, a mast cell, a neutrophil or a cell line derived therefrom.

51. The method of claim 40, wherein the single cell comprises an embryonic stem cell, an adult stem cell, or an iPS cell.

52. The method of claim 40, wherein the single cell further comprises a nucleic acid encoding a CRISPR enzyme.

53. A method of screening for modulators of secretion comprising:
a) contacting a cell comprising a non-naturally occurring nucleic acid construct with a test compound in the presence of a cell-impermeable marker capable of binding to a SNAP tag, wherein the non-naturally occurring nucleic acid construct encodes a fusion protein, wherein the encoded fusion protein: (i) is capable of being expressed in the single cell; (ii) is a vesicle membrane protein which has at least 90% identity to the amino acid sequence of VAMP1, VAMP2, VAMP3, VAMP4, VAMP5, VAMP7, VAMP8, synaptophysin or a synaptotagmin family protein, wherein the vesicle membrane protein comprises a cytoplasmic domain, a transmembrane domain and a vesicular domain, wherein the vesicular domain comprises the SNAP tag; and (iii) localizes to the membrane of a secretory vesicle such that the SNAP tag localizes to the lumen of the secretory vesicle, wherein the SNAP tag is capable of binding to the cell-impermeable marker upon secretion of the secretory vesicle; and
b) determining fluorescence of the cell,
whereby a difference in fluorescence as compared to the cell not contacted with a test compound indicates that the test compound is a modulator of secretion.

54. The method of claim 53, further comprising treating the cell with a secretagogue.

55. The method of claim 53, wherein determining fluorescence of single cells is by cell sorting.

56. The method of claim 53, wherein the test compound is a test nucleic acid, optionally wherein the test nucleic acid comprises a unique barcode sequence and/or the test nucleic acid comprises a CRISPR guide RNA, RNAi or gene expression sequence.

57. A kit comprising a non-naturally occurring nucleic acid construct, a cell-impermeable marker capable of binding to a SNAP tag, and instructions for its use, wherein the non-naturally occurring nucleic acid construct encodes a fusion protein, wherein the encoded fusion protein: (i) is capable of being expressed in a single cell; (ii) is a vesicle membrane protein which has at least 90% identity to the amino acid sequence of VAMP1, VAMP2, VAMP3, VAMP4, VAMP5, VAMP7, VAMP8, synaptophysin or a synaptotagmin family protein, wherein the vesicle membrane protein comprises a cytoplasmic domain, a transmembrane domain and a vesicular domain, wherein the vesicular domain comprises the SNAP tag; and (iii) localizes to the membrane of a secretory vesicle such that the SNAP tag localizes to the lumen of the secretory vesicle, wherein the SNAP tag is capable of binding to the cell-impermeable marker upon secretion of the secretory vesicle.

58. The kit of claim 57, further comprising at least one nucleic acid construct encoding a CRISPR guide RNA.
